(19) United States
(12) Patent Application Publication
(10) Pub. No.: US 2024/0335503 A1
(43) Pub. Date: Oct. 10, 2024

(54) ROMIDEPSIN AS A THERAPEUTIC AGENT FOR NERVE-INJURY INDUCED NEUROPATHIC PAIN AND SPASTICITY

(71) Applicant: The United States Government as represented by the Department of Veterans Affairs, Washington, DC (US)

(72) Inventor: Andrew Tan, Bethany, CT (US)

(21) Appl. No.: 18/579,252

(22) PCT Filed: Jul. 15, 2022
(86) PCT No.: PCT/US2022/037390

§ 371 Date: Jan. 12, 2024

Related U.S. Application Data
(60) Provisional application No. 63/222,305, filed on Jul. 15, 2021.

(51) Int. Cl. A61K 38/15; A61K 45/06; A61P 25/02; A61P 25/04

(52) U.S. Cl. CPC A61K 38/15; A61K 45/06; A61P 25/02; A61P 25/04

(57) ABSTRACT

The invention generally relates to methods of treating spasticity and/or neuropathic pain using known romidepsin and pharmaceutical compositions comprising same. This abstract is intended as a scanning tool for purposes of searching in the particular art and is not intended to be limiting of the present invention.

### CROSS-REFERENCE TO RELATED APPLICATIONS

This application claims the benefit of U.S. Application No. 63/222,305, filed on Jul. 15, 2021, the contents of which are incorporated herein by reference in their entirety.

### BACKGROUND

Spasticity is a clinical symptom of hyperexcitability within the spinal stretch reflex system (or H-reflex), which presents as a velocity-dependent increase in tonic stretch reflexes with exaggerated tendon jerks (Lance, 1980). The Hoffman (H)-reflex system is a simple circuit—signals along Ia sensory afferents from muscle spindles (which detect tissue stretch) project and synapse on spinal α-motor neurons, which drive muscle contraction. In chronic SCI, spasticity often presents below the injury as uncontrollable “jerking” movement and abnormal muscle tone whereby muscles continually contract (Skold et al., 1999). In animal models of SCI, reduced reflex control (during spinal shock) is followed by the development of spasticity, which often peaks 3 weeks post-SCI with elevated reflex excitability persisting for much longer (Bandaru et al., 2015; Eaton, 2003; Kitzman, 2007; Li et al., 2004). Along with other contributing factors to spasticity, such as the loss of supraspinal or local inhibitory input and dysfunctional cation conductance, SCI-induced structural plasticity can powerfully and adversely affect reflex function (Boulenguez et al., 2010; Fouad et al., 2013; Hultborn et al., 2007; Li et al., 2004; Nielsen et al., 2007; Raisman, 1994).

Dendritic spines are micron-sized, postsynaptic structures that contribute to modifying synaptic transmission and circuit function (Calabrese et al., 2006). In clinical investigations, post-mortem studies have revealed malformed dendritic spines (dysgenesis) in a spectrum of neuropsychiatric disorders, including PTSD, bipolar disorder, anxiety, and addiction (Halpain et al., 2005; Tan, 2015a; Tan, 2015b). Importantly, work over the previous decade has identified a common structural motif of dendritic spine morphology strongly associated with neuropathic pain and spasticity (Bandaru et al., 2015; Tan et al., 2012b; Tan et al., 2015b; Zhao et al., 2016) (FIG. 1). Specifically, dendritic spine profiles on spinal α-motor neurons associated with spasticity include: I) increased dendritic spine density, II) a redistribution of spines to regions closer to the cell body, and III) increased spine head surface area (Bandaru et al., 2015; Benson et al., 2017). It has also been shown that these particular dendritic spine profiles can increase neuronal circuit excitability in silico, and in a manner that can reproduce the physiological signs and symptoms in vivo (Tan et al., 2009).

Rac1 is a 21 kDa soluble intracellular protein that “switches” between an active or inactive state (i.e., Rac1 GTP-bound versus GDP-bound). In the hippocampus, constitutively active Rac1 increases dendritic spine density, stability, and volume; whereas, dominant-negative Rac1 (mutant RacN17 expression) decreases spine density, and inhibits spine maturation (Nakayama et al., 2000; Tashiro et al., 2004). Consistent with this, it has been demonstrated that Rac1 inhibition in vitro can attenuate the presence and maturation of dendritic spines in primary spinal cord neuron cultures (Tan et al., 2011; Zhao et al., 2016). Moreover, in a time-course longitudinal study, it has been shown that in vivo Rac1 activity is necessary and sufficient for dendritic spine dysgenesis on spinal α-motor neurons and spasticity after SCI (Bandaru et al., 2015) (FIG. 2). Intrathecal delivery of a Rac1-inhibitor drug, NSC23766, reduced the presence of abnormal dendritic spine profiles in α-motor neurons (ventral horn lamina IX) of SCI animals, and reduced excessive H-reflex excitability (e.g., restored RDD, reduced H/M ratio). Cessation of drug treatment resulted in a rapid return of both abnormal dendritic spine profiles and spasticity. The maximum-tolerated dose (MTD) in these studies did not affect baseline pain threshold, gross locomotor function, or significantly change dendritic spines in motor neuron pools of uninjured animals. Despite these promising results, Rac1 remains a problematic therapeutic target. Rac1 belongs to the Ras superfamily, a class of GTPases generally excluded from clinical development due to their complex intracellular dynamics (US clinical trials survey, accessed online Oct. 20, 2020).

Substantial evidence has identified PAK1 as a promising clinical target in cancer and cognitive dysfunction (Bertino et al., 2011; Kichina et al., 2010), and is involved in SCI-induced complications, i.e., hypoxia. Moreover, PAK1 is required for dendritic spine dysgenesis associated with many neuropsychiatric diseases (Baker-Herman et al., 2004; Boda et al., 2008; Hayashi et al., 2007; Liu et al., 2009; Ma et al., 2012). Although PAK1 has been implicated in mechanisms underlying pain (Asrar et al., 2009; Gao et al., 2004; Kichina et al., 2010; Wang et al., 2011), PAK1 as a potential therapeutic target for neuropathic pain and other disorders having a common structural motif of dendritic spine morphology (i.e., spasticity) is largely unexplored. Thus, there remains a need for compounds, compositions, and methods of treating neuropathic pain and spasticity.

### SUMMARY

In accordance with the purpose(s) of the invention, as embodied and broadly described herein, the invention, in one aspect, relates to methods of treating neuropathic pain and spasticity using romidepsin and pharmaceutical compositions comprising same.

Disclosed are methods for treating spasticity in a subject in need thereof, the method comprising administering to the subject an effective amount of a compound having a structure:

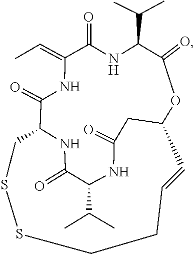 [SMILES: C/C=C1\NC(=O)[C@H]2CSSCC/C=C/[C@H](CC(=O)N[C@H](C(C)C)C(=O)N2)OC(=O)[C@H](C(C)C)NC1=O]

or a pharmaceutically acceptable salt thereof, thereby treating the subject for spasticity.

Also disclosed are methods for treating neuropathic pain in a subject in need thereof, the method comprising administering to the subject an effective amount of a compound having a structure:

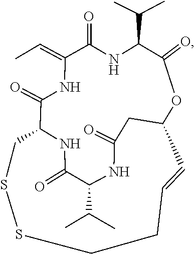 [SMILES: C/C=C1\NC(=O)[C@H]2CSSCC/C=C/[C@H](CC(=O)N[C@H](C(C)C)C(=O)N2)OC(=O)[C@H](C(C)C)NC1=O]

or a pharmaceutically acceptable salt thereof, thereby treating the subject for neuropathic pain.

Also disclosed are pharmaceutical compositions comprising an effective amount of a compound having a structure:

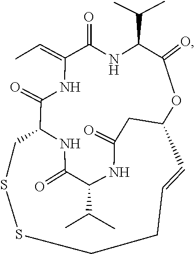 [SMILES: C/C=C1\NC(=O)[C@H]2CSSCC/C=C/[C@H](CC(=O)N[C@H](C(C)C)C(=O)N2)OC(=O)[C@H](C(C)C)NC1=O]

or a pharmaceutically acceptable salt thereof, and an effective amount of one or more of: (a) an agent known to treat spasticity; (b) an agent known to treat pain; and (c) a chemotherapeutic agent, and a pharmaceutically acceptable carrier.

Also disclosed kits comprising an effective amount of a compound of a compound having a structure:

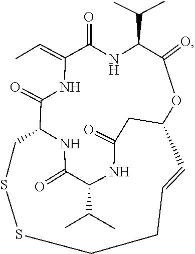 [SMILES: C/C=C1\NC(=O)[C@H]2CSSCC/C=C/[C@H](CC(=O)N[C@H](C(C)C)C(=O)N2)OC(=O)[C@H](C(C)C)NC1=O]

or a pharmaceutically acceptable salt thereof, and one or more of: (a) an agent known to treat spasticity; (b) an agent known to treat pain; (c) a chemotherapeutic agent; (d) instructions for treating spasticity; (e) instructions for treating pain; (f) instructions for administering the compound in connection with treating spasticity; and (g) instructions for administering the compound in connection with treating pain.

While aspects of the present invention can be described and claimed in a particular statutory class, such as the system statutory class, this is for convenience only and one of skill in the art will understand that each aspect of the present invention can be described and claimed in any statutory class. Unless otherwise expressly stated, it is in no way intended that any method or aspect set forth herein be construed as requiring that its steps be performed in a specific order. Accordingly, where a method claim does not specifically state in the claims or descriptions that the steps are to be limited to a specific order, it is no way intended that an order be inferred, in any respect. This holds for any possible non-express basis for interpretation, including matters of logic with respect to arrangement of steps or operational flow, plain meaning derived from grammatical organization or punctuation, or the number or type of aspects described in the specification.

### BRIEF DESCRIPTION OF THE DRAWINGS

The accompanying figures, which are incorporated in and constitute a part of this specification, illustrate several aspects and together with the description serve to explain the principles of the invention.

FIG. 1 shows a representative image illustrating that dendritic spine dysgenesis on α-motor neurons is associated with spasticity (Bandaru et al., 2015).

FIG. 2 shows representative data illustrating that Rac1 inhibition can reverse dendritic spine dysgenesis on α-motor neurons after SCI (Bandaru et al., 2015).

FIG. 3 shows a representative image illustrating Rac1 within an astrocyte-neuron model of spasticity in SCI/D.

FIG. 4A-D show representative data illustrating that Rac1 activity regulates dendritic spine dysgenesis in α-motor neurons and spasticity after SCI. Referring to FIG. 4A, 3D-neuronal analysis reveals dendritic spine dysgenesis after SCI. Referring to FIG. 4B and FIG. 4C, EMG recordings show an SCI-induced increase in evoked H-reflex excitability, shown with a loss of RDD. Referring to FIG. 4D, Rac1-inhibition reverses SCI-induced pathophysiology (Bandaru et al., 2015).

FIG. 5 shows representative data illustrating CTB labeled α-motor neurons (green) and VGLUT1-positive primary afferent boutons (red; white arrows) (Tan et al., 2012; Bandaru et al., 2015).

FIG. 6A and FIG. 6B show representative data illustrating that intramuscularly injected AAV-Cre reduces abnormal H-reflex response after SCI (in panel B: *=p<0.05; group mean SCI before vs. after SCI after AAV Cre injection; n=18 recordings per comparison; Mann-Whitney rank sum test).

FIG. 7A-C show representative data illustrating that tissue clearing allows whole spinal cord imaging (SWITCH protocol) (Murray et al., 2015). Visible tomato-reporter expression in AAV-Cre infected spinal cord motor neurons.

FIG. 8 shows representative images illustrating that dendritic spines are clearly visible in infected ventral spinal cord MNs expressing tdTomato reporter from SWITCH “cleared” spinal tissue (red arrows).

FIG. 9A-D show representative data illustrating the development of a viable colony of transgenic mice lacking astrocytic Rac1. Referring to FIG. 9A and FIG. 9B, 3-weeks post-SCI, Rac1 knockout animals appear to have decreased astrocyte hypertrophic response (L4 spinal cord; asterisks denote lamina IX of the ventral horn) (n=3 animals/group; graph not shown). Referring to the inset in FIG. 9B, GFAP-driven cre expression is revealed through tdTomato reporter expression (red), which co-localizes with GFAP immunolabeling (green). Referring to FIG. 9C, α-motor neuron dendritic spines and astrocytic processes (*) in confocal z-stacks of cleared spinal tissue are shown. Referring to FIG. 9D, SCI animals with astrocyte Rac1 (−/−) knockout appear to have an RDI) slope closer to baseline (less excitability) than compared with control SCI (more excitability).

FIG. 10A-C show representative data illustrating validation of shRNA knockdown constructs towards targeting Rac1. Referring to FIG. 10A, shRNA-Rac1 significantly reduces expression of mRNA in a neuronal cell line (ND7/23) as compared with controls. Referring to FIG. 10B, AAV viral delivery of shRNA reduces in Rac1 expression in infected cells in vitro. Referring to FIG. 10C, an infected motor neuron with dendritic spines express construct reporter protein, GFP, in vivo.

FIG. 11 shows representative data illustrating that dendritic spine density decreases with IPA3 treatment (*=p<0.05; T-test).

FIG. 12A and FIG. 12B show representative data illustrating that romidepsin MTD does not affect (FIG. 12A) body weight or (FIG. 12B) paw-grip strength.

FIG. 13 shows representative H-reflex recordings illustrating a partial reduction in spasticity (increased slope) with romidepsin treatment in SCI (n=12 recordings per group; *=p<0.05; Mann-Whitney rank sum).

FIG. 14A-C shows representative data illustrating that romidepsin has bioavailability in the spinal cord, as shown with H3 co-labeling with neurons (n=9-11 sections/group; *=p<0.05; one-way ANOVA).

FIG. 15A and FIG. 15B show representative H-reflex data illustrating significant restoration of rate-dependent depression (RDD) following SCI with romidepsin treatment.

FIG. 16 shows representative data illustrating that p-Raf expression decreases with romidepsin exposure within the spinal cord.

FIG. 17 shows representative H3 expression data illustrating the effectiveness of romidepsin to act within the spinal cord tissues of interest, thereby indicating the expected mode-of-action of the PAK1-inhibitor drug.

FIG. 18 shows a representative study design to determine the effectiveness of conditional Rac1 knockout in spinal cord α-motor neurons to relieve dendritic spine dysgenesis and spasticity after SCI.

FIG. 19 shows a representative image illustrating SWITCH tissue clearing with multiple immunoreactivity stains.

FIG. 20 shows a representative image illustrating that astrocytes are an integral tissue component in synaptic and dendritic spine function (Perez-Alvarez et al., 2014; Scholz et al., 2007).

FIG. 21 shows a representative study design to study the combined effect of Rac1 knockout in both motor neurons and astrocytes after SCI.

FIG. 22 shows a representative study design for assessing AAV-delivery of shRNA-knockdown of Rac1.

FIG. 23 shows a representative schematic illustrating that the signaling pathway of PAK1 regulates dendritic spine dynamics. PAK1-inhibitors include FK228 (Romidepsin) and IPA3 (adapted from Nikolic, 2008).

FIG. 24 shows a representative romidepsin “cross-over” study design.

FIG. 25 shows representative data illustrating the bioavailability of romidepsin injection. Tissue exposure in the spinal cord results in an upregulation in acetyl histone-H3 in neurons. Analysis of co-labeled yellow puncta in the dorsal horn (spinal level L4) (Guo et al., 2018).

FIG. 26 shows a representative study design with time points to assess the efficacy of romidepsin for addressing nerve injury-induced chronic pain.

FIG. 27 shows representative data illustrating that romidepsin administration following a peripheral nerve injury significantly decreases mechanical-evoked pain.

FIG. 28 shows a representative study design with time points to assess the efficacy of romidepsin for addressing nerve injury-induced neuropathic pain.

FIG. 29 shows representative methodology for in vivo structural analysis of romidepsin-effect in the nociceptive system of the spinal cord following SNI, and behavioral/functional assessment for tactile and heat-pain threshold (Benson et al., 2020).

FIG. 30 shows a representative image of a Bioseb ActivMeter device.

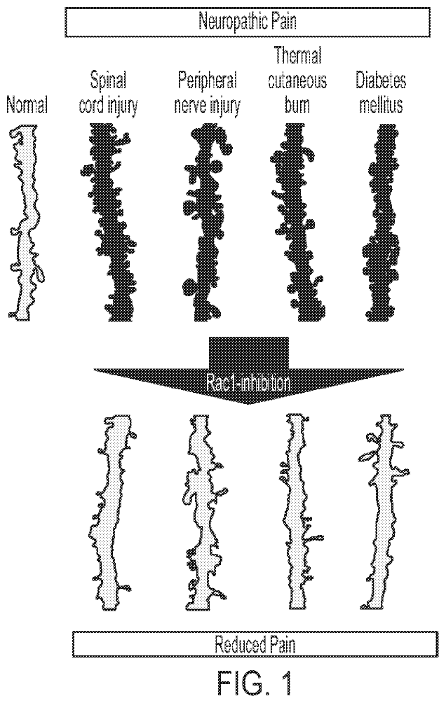

Additional advantages of the invention will be set forth in part in the description which follows, and in part will be obvious from the description, or can be learned by practice of the invention. The advantages of the invention will be realized and attained by means of the elements and combinations particularly pointed out in the appended claims. It is to be understood that both the foregoing general description and the following detailed description are exemplary and explanatory only and are not restrictive of the invention, as claimed.

### DETAILED DESCRIPTION

The present invention can be understood more readily by reference to the following detailed description of the invention and the Examples included therein.

Before the present compounds, compositions, articles, systems, devices, and/or methods are disclosed and described, it is to be understood that they are not limited to specific synthetic methods unless otherwise specified, or to particular reagents unless otherwise specified, as such may, of course, vary. It is also to be understood that the terminology used herein is for the purpose of describing particular aspects only and is not intended to be limiting. Although any methods and materials similar or equivalent to those described herein can be used in the practice or testing of the present invention, example methods and materials are now described.

While aspects of the present invention can be described and claimed in a particular statutory class, such as the system statutory class, this is for convenience only and one of skill in the art will understand that each aspect of the present invention can be described and claimed in any statutory class. Unless otherwise expressly stated, it is in no way intended that any method or aspect set forth herein be construed as requiring that its steps be performed in a specific order. Accordingly, where a method claim does not specifically state in the claims or descriptions that the steps are to be limited to a specific order, it is no way intended that an order be inferred, in any respect. This holds for any possible non-express basis for interpretation, including matters of logic with respect to arrangement of steps or operational flow, plain meaning derived from grammatical organization or punctuation, or the number or type of aspects described in the specification.

Throughout this application, various publications are referenced. The disclosures of these publications in their entireties are hereby incorporated by reference into this application in order to more fully describe the state of the art to which this pertains. The references disclosed are also individually and specifically incorporated by reference herein for the material contained in them that is discussed in the sentence in which the reference is relied upon. Nothing herein is to be construed as an admission that the present invention is not entitled to antedate such publication by virtue of prior invention. Further, the dates of publication provided herein may be different from the actual publication dates, which can require independent confirmation.

### A. DEFINITIONS

As used in the specification and the appended claims, the singular forms “a,” “an” and “the” include plural referents unless the context clearly dictates otherwise. Thus, for example, reference to “a functional group,” “an alkyl,” or “a residue” includes mixtures of two or more such functional groups, alkyls, or residues, and the like.

Ranges can be expressed herein as from “about” one particular value, and/or to “about” another particular value. When such a range is expressed, another aspect includes from the one particular value and/or to the other particular value. Similarly, when values are expressed as approximations, by use of the antecedent “about,” it will be understood that the particular value forms another aspect. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that each unit between two particular units are also disclosed. For example, if 10 and 15 are disclosed, then 11, 12, 13, and 14 are also disclosed.

References in the specification and concluding claims to parts by weight of a particular element or component in a composition denotes the weight relationship between the element or component and any other elements or components in the composition or article for which a part by weight is expressed. Thus, in a compound containing 2 parts by weight of component X and 5 parts by weight component Y, X and Y are present at a weight ratio of 2: 5, and are present in such ratio regardless of whether additional components are contained in the compound.

A weight percent (wt. %) of a component, unless specifically stated to the contrary, is based on the total weight of the formulation or composition in which the component is included.

As used herein, the terms “optional” or “optionally” means that the subsequently described event or circumstance can or cannot occur, and that the description includes instances where said event or circumstance occurs and instances where it does not.

As used herein, the term “subject” can be a vertebrate, such as a mammal, a fish, a bird, a reptile, or an amphibian. Thus, the subject of the herein disclosed methods can be a human, non-human primate, horse, pig, rabbit, dog, sheep, goat, cow, cat, guinea pig or rodent. The term does not denote a particular age or sex. Thus, adult and new born subjects, as well as fetuses, whether male or female, are intended to be covered. In one aspect, the subject is a mammal. A patient refers to a subject afflicted with a disease or disorder. The term “patient” includes human and veterinary subjects. In some aspects of the disclosed methods, the subject has been diagnosed with a need for treatment of one or more disorders prior to the administering step. In various aspects, the one or more disorders are an influenza viral infection.

As used herein, the term “treatment” refers to the medical management of a patient with the intent to cure, ameliorate, stabilize, or prevent a disease, pathological condition, or disorder. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder. In various aspects, the term covers any treatment of a subject, including a mammal (e.g., a human), and includes; (i) preventing the disease from occurring in a subject that can be predisposed to the disease but has not yet been diagnosed as having it; (ii) inhibiting the disease, i.e., arresting its development; or (iii) relieving the disease, i.e., causing regression of the disease. In one aspect, the subject is a mammal such as a primate, and, in a further aspect, the subject is a human. The term “subject” also includes domesticated animals (e.g., cats, dogs, etc.), livestock (e.g., cattle, horses, pigs, sheep, goats, etc.), and laboratory animals (e.g., mouse, rabbit, rat, guinea pig, fruit fly, etc.).

As used herein, the term “prevent” or “preventing” refers to precluding, averting, obviating, forestalling, stopping, or hindering something from happening, especially by advance action. It is understood that where reduce, inhibit or prevent are used herein, unless specifically indicated otherwise, the use of the other two words is also expressly disclosed.

As used herein, the term “diagnosed” means having been subjected to a physical examination by a person of skill, for example, a physician, and found to have a condition that can be diagnosed or treated by the compounds, compositions, or methods disclosed herein. In some aspects of the disclosed methods, the subject has been diagnosed with a need for treatment of a viral infection prior to the administering step. As used herein, the phrase “identified to be in need of treatment for a disorder,” or the like, refers to selection of a subject based upon need for treatment of the disorder. It is contemplated that the identification can, in one aspect, be performed by a person different from the person making the diagnosis. It is also contemplated, in a further aspect, that the administration can be performed by one who subsequently performed the administration.

As used herein, the terms “administering” and “administration” refer to any method of providing a pharmaceutical preparation to a subject. Such methods are well known to those skilled in the art and include, but are not limited to, oral administration, transdermal administration, administration by inhalation, nasal administration, topical administration, intravaginal administration, ophthalmic administration, intraaural administration, intracerebral administration, rectal administration, and parenteral administration, including injectable such as intravenous administration, intra-arterial administration, intramuscular administration, and subcutaneous administration. Administration can be continuous or intermittent. In various aspects, a preparation can be administered therapeutically; that is, administered to treat an existing disease or condition. In further various aspects, a preparation can be administered prophylactically; that is, administered for prevention of a disease or condition.

The term “contacting” as used herein refers to bringing a disclosed compound and a cell, target receptor, or other biological entity together in such a manner that the compound can affect the activity of the target (e.g., receptor, cell, etc.), either directly; i.e., by interacting with the target itself, or indirectly; i.e., by interacting with another molecule, co-factor, factor, or protein on which the activity of the target is dependent.

As used herein, the terms “effective amount” and “amount effective” refer to an amount that is sufficient to achieve the desired result or to have an effect on an undesired condition. For example, a “therapeutically effective amount” refers to an amount that is sufficient to achieve the desired therapeutic result or to have an effect on undesired symptoms, but is generally insufficient to cause adverse side effects. The specific therapeutically effective dose level for any particular patient will depend upon a variety of factors including the disorder being treated and the severity of the disorder: the specific composition employed; the age, body weight, general health, sex and diet of the patient; the time of administration; the route of administration; the rate of excretion of the specific compound employed; the duration of the treatment; drugs used in combination or coincidental with the specific compound employed and like factors well known in the medical arts. For example, it is well within the skill of the art to start doses of a compound at levels lower than those required to achieve the desired therapeutic effect and to gradually increase the dosage until the desired effect is achieved. If desired, the effective daily dose can be divided into multiple doses for purposes of administration. Consequently, single dose compositions can contain such amounts or submultiples thereof to make up the daily dose. The dosage can be adjusted by the individual physician in the event of any contraindications. Dosage can vary, and can be administered in one or more dose administrations daily, for one or several days. Guidance can be found in the literature for appropriate dosages for given classes of pharmaceutical products. In further various aspects, a preparation can be administered in a “prophylactically effective amount”; that is, an amount effective for prevention of a disease or condition.

As used herein, “IC₅₀” is intended to refer to the concentration of a substance (e.g., a compound or a drug) that is required for 50% inhibition of a biological process, or component of a process, including a protein, subunit, organelle, ribonucleoprotein, etc. In one aspect, an IC₅₀ can refer to the concentration of a substance that is required for 50% inhibition in vivo, as further defined elsewhere herein. In a further aspect, IC₅₀ refers to the half maximal (50%) inhibitory concentration (IC) of a substance.

As used herein, “EC₅₀” is intended to refer to the concentration of a substance (e.g., a compound or a drug) that is required for 50% agonism of a biological process, or component of a process, including a protein, subunit, organelle, ribonucleoprotein, etc. In one aspect, an EC₅₀ can refer to the concentration of a substance that is required for 50% agonism in vivo, as further defined elsewhere herein. In a further aspect, EC₅₀ refers to the concentration of agonist that provokes a response halfway between the baseline and maximum response.

The term “pharmaceutically acceptable” describes a material that is not biologically or otherwise undesirable, i.e., without causing an unacceptable level of undesirable biological effects or interacting in a deleterious manner.

As used herein, the term “derivative” refers to a compound having a structure derived from the structure of a parent compound (e.g., a compound disclosed herein) and whose structure is sufficiently similar to those disclosed herein and based upon that similarity, would be expected by one skilled in the art to exhibit the same or similar activities and utilities as the claimed compounds, or to induce, as a precursor, the same or similar activities and utilities as the claimed compounds. Exemplary derivatives include salts, esters, amides, salts of esters or amides, and N-oxides of a parent compound.

Compounds described herein may comprise atoms in both their natural isotopic abundance and in non-natural abundance. The disclosed compounds can be isotopically labeled or isotopically substituted compounds identical to those described, but for the fact that one or more atoms are replaced by an atom having an atomic mass or mass number different from the atomic mass or mass number typically found in nature. Examples of isotopes that can be incorporated into compounds of the invention include isotopes of hydrogen, carbon, nitrogen, oxygen, phosphorous, fluorine and chlorine, such as ²H, ³H, ¹³C, ¹⁴C, ¹⁵N, ¹⁸O, ¹⁷O, ³⁵S, ¹⁸F and ³⁶Cl, respectively. Compounds further comprise prodrugs thereof, and pharmaceutically acceptable salts of said compounds or of said prodrugs which contain the aforementioned isotopes and/or other isotopes of other atoms are within the scope of this invention. Certain isotopically labeled compounds of the present invention, for example those into which radioactive isotopes such as ³H and ¹⁴C are incorporated, are useful in drug and/or substrate tissue distribution assays. Tritiated, i.e., ³H, and carbon-14, i.e., ¹⁴C, isotopes are particularly preferred for their ease of preparation and detectability. Further, substitution with heavier isotopes such as deuterium, i.e., ²H, can afford certain therapeutic advantages resulting from greater metabolic stability, for example increased in vivo half-life or reduced dosage requirements and, hence, may be preferred in some circumstances. Isotopically labeled compounds of the present invention and prodrugs thereof can generally be prepared by carrying out the procedures below, by substituting a readily available isotopically labeled reagent for a non-isotopically labeled reagent.

The compounds described in the invention can be present as a solvate. In some cases, the solvent used to prepare the solvate is an aqueous solution, and the solvate is then often referred to as a hydrate. The compounds can be present as a hydrate, which can be obtained, for example, by crystallization from a solvent or from aqueous solution. In this connection, one, two, three or any arbitrary number of solvate or water molecules can combine with the compounds according to the invention to form solvates and hydrates. Unless stated to the contrary, the invention includes all such possible solvates.

The term “co-crystal” means a physical association of two or more molecules that owe their stability through non-covalent interaction. One or more components of this molecular complex provide a stable framework in the crystalline lattice. In certain instances, the guest molecules are incorporated in the crystalline lattice as anhydrates or solvates, see e.g., Almarasson, O., et al. (2004) The Royal Society of Chemistry, 1889-1896. Examples of co-crystals include p-toluenesulfonic acid and benzenesulfonic acid.

It is known that chemical substances form solids that are present in different states of order that are termed polymorphic forms or modifications. The different modifications of a polymorphic substance can differ greatly in their physical properties. The compounds according to the invention can be present in different polymorphic forms, with it being possible for particular modifications to be metastable. Unless stated to the contrary, the invention includes all such possible polymorphic forms.

Certain materials, compounds, compositions, and components disclosed herein can be obtained commercially or readily synthesized using techniques generally known to those of skill in the art. For example, the starting materials and reagents used in preparing the disclosed compounds and compositions are either available from commercial suppliers such as Aldrich Chemical Co., (Milwaukee, Wis.), Acros Organics (Morris Plains, N.J.), Fisher Scientific (Pittsburgh, Pa.), or Sigma (St. Louis, Mo.) or are prepared by methods known to those skilled in the art following procedures set forth in references such as Fieser and Fieser's Reagents for Organic Synthesis, Volumes 1-17 (John Wiley and Sons, 1991); Rodd's Chemistry of Carbon Compounds, Volumes 1-5 and Supplementals (Elsevier Science Publishers, 1989); Organic Reactions, Volumes 1-40 (John Wiley and Sons, 1991); March's Advanced Organic Chemistry, (John Wiley and Sons, 4th Edition); and Larock's Comprehensive Organic Transformations (VCH Publishers Inc., 1989).

Unless otherwise expressly stated, it is in no way intended that any method set forth herein be construed as requiring that its steps be performed in a specific order. Accordingly, where a method claim does not actually recite an order to be followed by its steps or it is not otherwise specifically stated in the claims or descriptions that the steps are to be limited to a specific order, it is no way intended that an order be inferred, in any respect. This holds for any possible non-express basis for interpretation, including: matters of logic with respect to arrangement of steps or operational flow; plain meaning derived from grammatical organization or punctuation; and the number or type of embodiments described in the specification.

It is understood that the compositions disclosed herein have certain functions. Disclosed herein are certain structural requirements for performing the disclosed functions, and it is understood that there are a variety of structures that can perform the same function that are related to the disclosed structures, and that these structures will typically achieve the same result.

### B. METHODS FOR TREATING SPASTICITY

In one aspect, disclosed are methods for treating spasticity in a subject in need thereof, the method comprising administering to the subject an effective amount of a compound having a structure:

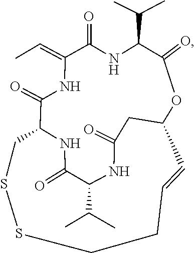 [SMILES: C/C=C1\NC(=O)[C@H]2CSSCC/C=C/[C@H](CC(=O)N[C@H](C(C)C)C(=O)N2)OC(=O)[C@H](C(C)C)NC1=O]

or a pharmaceutically acceptable salt thereof, thereby treating the subject for spasticity. In a further aspect, spasticity is induced by spinal cord injury (SCI) or multiple scelorsis (MS).

To treat or control the disorder, the compounds and pharmaceutical compositions comprising the compounds are administered to a subject in need thereof, such as a vertebrate, e.g., a mammal, a fish, a bird, a reptile, or an amphibian. The subject can be a human, non-human primate, horse, pig, rabbit, dog, sheep, goat, cow, cat, guinea pig or rodent. The term does not denote a particular age or sex. Thus, adult and newborn subjects, as well as fetuses, whether male or female, are intended to be covered. The subject is preferably a mammal, such as a human. Prior to administering the compounds or compositions, the subject can be diagnosed with a need for treatment of spasticity, such as, for example, spasticity induced by spinal cord injury or multiple scelorsis (MS).

The compounds or compositions can be administered to the subject according to any method. Such methods are well known to those skilled in the art and include, but are not limited to, oral administration, transdermal administration, administration by inhalation, nasal administration, topical administration, intravaginal administration, ophthalmic administration, intraaural administration, intracerebral administration, intrathecal administration, rectal administration, sublingual administration, buccal administration and parenteral administration, including injectable such as intravenous administration, intra-arterial administration, intramuscular administration, and subcutaneous administration. Administration can be continuous or intermittent. A preparation can be administered therapeutically; that is, administered to treat an existing disease or condition. A preparation can also be administered prophylactically; that is, administered for prevention of spasticity, such as, for example, spasticity induced by spinal cord injury or multiple scelorsis (MS).

The therapeutically effective amount or dosage of the compound can vary within wide limits. Such a dosage is adjusted to the individual requirements in each particular case including the specific compound(s) being administered, the route of administration, the condition being treated, as well as the patient being treated. In general, in the case of oral or parenteral administration to adult humans weighing approximately 70 Kg or more, a daily dosage of about 10 mg to about 10,000 mg, preferably from about 200 mg to about 1,000 mg, should be appropriate, although the upper limit may be exceeded. The daily dosage can be administered as a single dose or in divided doses, or for parenteral administration, as a continuous infusion. Single dose compositions can contain such amounts or submultiples thereof of the compound or composition to make up the daily dose. The dosage can be adjusted by the individual physician in the event of any contraindications. Dosage can vary, and can be administered in one or more dose administrations daily, for one or several days.

In a further aspect, the subject is not currently undergoing chemotherapy. In a still further aspect, the subject has not previously undergone chemotherapy within the last 7 days. In yet a further aspect, the subject has not previously undergone chemotherapy within the last 14 days. In an even further aspect, the subject has not previously undergone chemotherapy within the last month.

In a further aspect, the subject is a mammal. In a still further aspect, the mammal is a human.

In a further aspect, the subject has been diagnosed with a need for treatment of spasticity prior to the administering step. In a still further aspect, the method further comprises the step of identifying a subject in need of treatment of spasticity.

In a further aspect, the effective amount is a therapeutically effective amount. In a still further aspect, the effective amount is a prophylactically effective amount.

In a further aspect, the effective amount is an amount of from about 0.1 mg/kg to about 0.4 mg/kg, about 0.1 mg/kg to about 0.3 mg/kg, about 0.1 mg/kg to about 0.2 mg/kg, about 0.2 mg/kg to about 0.4 mg/kg, about 0.3 mg/kg to about 0.4 mg/kg, or about 0.2 mg/kg to about 0.3 mg/kg. In a still further aspect, the effective amount is an amount of about 0.1 mg/kg, about 0.15 mg/kg, about 0.2 mg/kg, about 0.25 mg/kg, about 0.3 mg/kg, about 0.35 mg/kg, or about 0.4 mg/kg. In yet a further aspect, the effective amount is an amount of about 0.25 mg/kg.

In a further aspect, the effective amount is administered once per day. In a still further aspect, the effective amount is administered once per day for a period of at least 2 days, at least 3 days, at least 4 days, at least 5 days, at least 6 days, at least 7 days, at least 8 days, at least 9 days, at least 10 days, at least 11 days, at least 12 days, at least 13 days, or at least 14 days. In yet a further aspect, the effective amount is administered once per day for a period of at least 7 days. In an even further aspect, the effective amount is administered once per day for a period of at least 14 days.

In a further aspect, administering is via systemic administration.

In a further aspect, the method further comprises administering to the subject an effective amount of an agent known to treat spasticity. Examples of agents known to treat spasticity include, but are not limited to, baclofen, tizanidine, dantrolene sodium, diazepam, clonazepam, and gabapentin. In a still further aspect, the compound and the agent are administered sequentially. In yet a further aspect, the compound and the agent are administered simultaneously. In an even further aspect, the compound and the agent are co-formulated. In a still further aspect, the compound and the agent are not co-formulated.

In a further aspect, the method further comprises administering to the subject an effective amount of an agent known to treat pain. Examples of agents known to treat pain include, but are not limited to, a nonsteroidal anti-inflammatory drug (NSAID) (e.g., flurbiprofen, ketorolac, ketoprofen, tolmetin, aspirin, ibuprofen, naproxen, indomethacin, sulindac, piroxicam, mefenamic acid, meloxicam, diclofenac, celecoxib, etodolac, etoricoxib, lumiracoxib, rofecoxib), an antimigraine agent (e.g., almotriptan, dihydroergotamine, eletriptan, ergotamine, frovatriptan, naratriptan, rizatriptan, sumatriptan, zomitriptan), a COX-2 inhibitor (e.g., celecoxib, rofecoxib, valdecoxib), acetaminophen, ziconotide, a narcotic (e.g., alfentanil, buprenorphine, butorphano, codeine, fentanyl, hydrocodone, hydromorphone, levorphanol, meperidine, methadone, morphine, nalbuphine, oxycodone, oxymorphone, propoxyphene, tramadol, tapentadol), and a salicylate (e.g., aspirin, diflunisal, magnesium salicylate, salsalate). In a still further aspect, the compound and the agent are administered sequentially. In yet a further aspect, the compound and the agent are administered simultaneously. In an even further aspect, the compound and the agent are co-formulated. In a still further aspect, the compound and the agent are not co-formulated. In yet a further aspect, pain is acute pain, chronic pain, or neuropathic pain. In an even further aspect, pain is neuropathic pain.

In a further aspect, the method further comprises administering to the subject an effective amount of a chemotherapeutic agent. Examples of chemotherapeutic agents include, but are not limited to, an alkylating agent (e.g., carboplatin, cisplatin, cyclophosphamide, chlorambucil, melphalan, carmustine, busulfan, lomustine, dacarbazine, oxaliplatin, ifosfamide, mechlorethamine, temozolomide, thiotepa, bendamustine, streptozocin), an antimetabolite agent (e.g., gemcitabine, 5-fluorouracil, capecitabine, hydroxyurea, mercaptopurine, pemetrexed, fludarabine, nelarabine, cladribine, clofarabine, cytarabine, decitabine, pralatrexate, floxuridine, methotrexate, thioguanine), an antineoplastic antibiotic agent (e.g., doxorubicin, mitoxantrone, bleomycin, daunorubicin, dactinomycin, epirubicin, idarubicin, plicamycin, mitomycin, pentostatin, valrubicin), a mitotic inhibitor agent (e.g., irinotecan, topotecan, rubitecan, cabazitaxel, docetaxel, paclitaxel, etopside, vincristine, ixabepilone, vinorelbine, vinblastine, teniposide), and a mTor inhibitor agent (e.g., everolimus, siroliumus, temsirolimus). In a still further aspect, the compound and the agent are administered sequentially. In yet a further aspect, the compound and the agent are administered simultaneously. In an even further aspect, the compound and the agent are co-formulated. In a still further aspect, the compound and the agent are not co-formulated.

### C. METHODS FOR TREATING NEUROPATHIC PAIN

In one aspect, disclosed are methods for treating neuropathic pain in a subject in need thereof, the method comprising administering to the subject an effective amount of a compound having a structure:

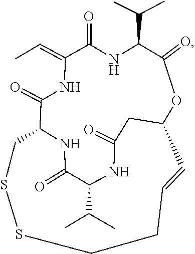 [SMILES: C/C=C1\NC(=O)[C@H]2CSSCC/C=C/[C@H](CC(=O)N[C@H](C(C)C)C(=O)N2)OC(=O)[C@H](C(C)C)NC1=O]

or a pharmaceutically acceptable salt thereof, thereby treating the subject for neuropathic pain. In a further aspect, the subject has a peripheral nerve injury.

To treat or control the disorder, the compounds and pharmaceutical compositions comprising the compounds are administered to a subject in need thereof, such as a vertebrate, e.g., a mammal, a fish, a bird, a reptile, or an amphibian. The subject can be a human, non-human primate, horse, pig, rabbit, dog, sheep, goat, cow, cat, guinea pig or rodent. The term does not denote a particular age or sex. Thus, adult and newborn subjects, as well as fetuses, whether male or female, are intended to be covered. The subject is preferably a mammal, such as a human. Prior to administering the compounds or compositions, the subject can be diagnosed with a need for treatment of neuropathic pain, such as, for example, neuropathic pain due to a peripheral nerve injury.

The compounds or compositions can be administered to the subject according to any method. Such methods are well known to those skilled in the art and include, but are not limited to, oral administration, transdermal administration, administration by inhalation, nasal administration, topical administration, intravaginal administration, ophthalmic administration, intraaural administration, intracerebral administration, intrathecal administration, rectal administration, sublingual administration, buccal administration and parenteral administration, including injectable such as intravenous administration, intra-arterial administration, intramuscular administration, and subcutaneous administration. Administration can be continuous or intermittent. A preparation can be administered therapeutically; that is, administered to treat an existing disease or condition. A preparation can also be administered prophylactically; that is, administered for prevention of neuropathic pain, such as, for example, neuropathic pain due to a peripheral nerve injury caused by amputation, surgical complication or trauma, or a disease (e.g., a metabolic disease).

The therapeutically effective amount or dosage of the compound can vary within wide limits. Such a dosage is adjusted to the individual requirements in each particular case including the specific compound(s) being administered, the route of administration, the condition being treated, as well as the patient being treated. In general, in the case of oral or parenteral administration to adult humans weighing approximately 70 Kg or more, a daily dosage of about 10 mg to about 10,000 mg, preferably from about 200 mg to about 1,000 mg, should be appropriate, although the upper limit may be exceeded. The daily dosage can be administered as a single dose or in divided doses, or for parenteral administration, as a continuous infusion. Single dose compositions can contain such amounts or submultiples thereof of the compound or composition to make up the daily dose. The dosage can be adjusted by the individual physician in the event of any contraindications. Dosage can vary, and can be administered in one or more dose administrations daily, for one or several days.

In a further aspect, the peripheral nerve injury is due to an injury. In a still further aspect, the injury is amputation or is due to surgical complication or trauma.

In a further aspect, the peripheral nerve injury is due to a disease. In a still further aspect, the disease is a metabolic disease. Examples of metabolic diseases include, but are not limited to, heart disease, diabetes, stroke, multiple sclerosis (MS) a lysosomal storage disease (e.g., Hurler syndrome, Gaucher disease, Niemann-Pick disease, Fabry disease, Tay-Sachs disease, a mucopolysaccharidoses (MPS) disease, Pompe disease), maple syrup urine disease, a glycogen storage disease (e.g., Von Gierke disease, Pompe's disease, Cori's disease. Andersen's disease, McArdle's disease, Hers™ disease, Tarui's disease), mitochondrial disease (e.g., mitochondrial myopathy, diabetes mellitus and deafness (DAD), Leber's hereditary optic neuropathy (LHON), Leigh syndrome, neuropathy, ataxia, retinitis pigmentosa, and ptosis (NARP), myoneurogenic gastrointestinal encephalopathy (MNGIE), MERRF syndrome, MELAS syndrome, mitochondrial DNA depletion syndrome), a peroxisomal disorder (e.g., X-linked adrenoleukodystrophy (X-ALD), Zellweger syndrome (ZS), neonatal adrenoleukodystrophy (NALD), infantile Refsum disease (IRD), rhizomelic chondrodysplasia punctata (RCDP), Zellweger-like syndrome), and a metal metabolism disorder (e.g., hypermanganesemia with dystonia 1, hypermanganesemia with dystonia 2, Wilson disease, acaeruloplasminaemia, cerebellar ataxia, hypoceruloplasminemia, neuroferritinopathy, hyperferritinemia-cataract syndrome, L-ferritin deficiency, spastic paraplegia, HARP syndrome, pontocerebellar hypoplasia, infantile neuroaxonal dystrophy, Parkinson's disease, Woohouse-Sakati syndrome, Kufor-Rakeb syndrome, hemochromatosis). In yet a further aspect, the metabolic disease is selected from diabetes and multiple sclerosis (MS).

In a further aspect, the subject is not currently undergoing chemotherapy. In a still further aspect, the subject has not previously undergone chemotherapy within the last 7 days. In yet a further aspect, the subject has not previously undergone chemotherapy within the last 14 days. In an even further aspect, the subject has not previously undergone chemotherapy within the last month.

In a further aspect, the subject is a mammal. In a still further aspect, the mammal is a human.

In a further aspect, the subject has been diagnosed with a need for treatment of neuropathic pain prior to the administering step. In a still further aspect, the method further comprises the step of identifying a subject in need of treatment of neuropathic pain.

In a further aspect, the effective amount is a therapeutically effective amount. In a still further aspect, the effective amount is a prophylactically effective amount.

In a further aspect, the effective amount is an amount of from about 0.1 mg/kg to about 0.4 mg/kg, about 0.1 mg/kg to about 0.3 mg/kg, about 0.1 mg/kg to about 0.2 mg/kg, about 0.2 mg/kg to about 0.4 mg/kg, about 0.3 mg/kg to about 0.4 mg/kg, or about 0.2 mg/kg to about 0.3 mg/kg. In a still further aspect, the effective amount is an amount of about 0.1 mg/kg, about 0.15 mg/kg, about 0.2 mg/kg, about 0.25 mg/kg, about 0.3 mg/kg, about 0.35 mg/kg, or about 0.4 mg/kg. In yet a further aspect, the effective amount is an amount of about 0.25 mg/kg.

In a further aspect, the effective amount is administered once per day. In a still further aspect, the effective amount is administered once per day for a period of at least 2 days, at least 3 days, at least 4 days, at least 5 days, at least 6 days, at least 7 days, at least 8 days, at least 9 days, at least 10 days, at least 11 days, at least 12 days, at least 13 days, or at least 14 days. In yet a further aspect, the effective amount is administered once per day for a period of at least 7 days. In an even further aspect, the effective amount is administered once per day for a period of at least 14 days.

In a further aspect, administering is via systemic administration.

In a further aspect, the method further comprises administering to the subject an effective amount of an agent known to treat spasticity. Examples of agents known to treat spasticity include, but are not limited to, baclofen, tizanidine, dantrolene sodium, diazepam, clonazepam, and gabapentin. In a still further aspect, the compound and the agent are administered sequentially. In yet a further aspect, the compound and the agent are administered simultaneously. In an even further aspect, the compound and the agent are co-formulated. In a still further aspect, the compound and the agent are not co-formulated.

In a further aspect, the method further comprises administering to the subject an effective amount of an agent known to treat pain. Examples of agents known to treat pain include, but are not limited to, a nonsteroidal anti-inflammatory drug (NSAID) (e.g., flurbiprofen, ketorolac, ketoprofen, tolmetin, aspirin, ibuprofen, naproxen, indomethacin, sulindac, piroxicam, mefenamic acid, meloxicam, diclofenac, celecoxib, etodolac, etoricoxib, lumiracoxib, rofecoxib), an antimigraine agent (e.g., almotriptan, dihydroergotamine, eletriptan, ergotamine, frovatriptan, naratriptan, rizatriptan, sumatriptan, zomitriptan), a COX-2 inhibitor (e.g., celecoxib, rofecoxib, valdecoxib), acetaminophen, ziconotide, a narcotic (e.g., alfentanil, buprenorphine, butorphano, codeine, fentanyl, hydrocodone, hydromorphone, levorphanol, meperidine, methadone, morphine, nalbuphine, oxycodone, oxymorphone, propoxyphene, tramadol, tapentadol), and a salicylate (e.g., aspirin, diflunisal, magnesium salicylate, salsalate). In a still further aspect, the compound and the agent are administered sequentially. In yet a further aspect, the compound and the agent are administered simultaneously. In an even further aspect, the compound and the agent are co-formulated. In a still further aspect, the compound and the agent are not co-formulated. In yet a further aspect, pain is acute pain, chronic pain, or neuropathic pain. In an even further aspect, pain is neuropathic pain.

In a further aspect, the method further comprises administering to the subject an effective amount of a chemotherapeutic agent. Examples of chemotherapeutic agents include, but are not limited to, an alkylating agent (e.g., carboplatin, cisplatin, cyclophosphamide, chlorambucil, melphalan, carmustine, busulfan, lomustine, dacarbazine, oxaliplatin, ifosfamide, mechlorethamine, temozolomide, thiotepa, bendamustine, streptozocin), an antimetabolite agent (e.g., gemcitabine, 5-fluorouracil, capecitabine, hydroxyurea, mercaptopurine, pemetrexed, fludarabine, nelarabine, cladribine, clofarabine, cytarabine, decitabine, pralatrexate, floxuridine, methotrexate, thioguanine), an antineoplastic antibiotic agent (e.g., doxorubicin, mitoxantrone, bleomycin, daunorubicin, dactinomycin, epirubicin, idarubicin, plicamycin, mitomycin, pentostatin, valrubicin), a mitotic inhibitor agent (e.g., irinotecan, topotecan, rubitecan, cabazitaxel, docetaxel, paclitaxel, etopside, vincristine, ixabepilone, vinorelbine, vinblastine, teniposide), and a mTor inhibitor agent (e.g., everolimus, siroliumus, temsirolimus). In a still further aspect, the compound and the agent are administered sequentially. In yet a further aspect, the compound and the agent are administered simultaneously. In an even further aspect, the compound and the agent are co-formulated. In a still further aspect, the compound and the agent are not co-formulated.

### D. PHARMACEUTICAL COMPOSITIONS

In one aspect, disclosed are pharmaceutical compositions comprising an effective amount of a compound having a structure:

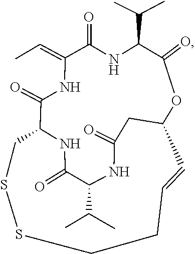 [SMILES: C/C=C1\NC(=O)[C@H]2CSSCC/C=C/[C@H](CC(=O)N[C@H](C(C)C)C(=O)N2)OC(=O)[C@H](C(C)C)NC1=O]

or a pharmaceutically acceptable salt thereof, and an effective amount of one or more of: (a) an agent known to treat spasticity; (b) an agent known to treat pain; and (c) a chemotherapeutic agent, and a pharmaceutically acceptable carrier.

Pharmaceutically acceptable salts of the compounds are conventional acid-addition salts or base-addition salts that retain the biological effectiveness and properties of the compounds and are formed from suitable non-toxic organic or inorganic acids or organic or inorganic bases. Exemplary acid-addition salts include those derived from inorganic acids such as hydrochloric acid, hydrobromic acid, hydroiodic acid, sulfuric acid, sulfamic acid, phosphoric acid and nitric acid, and those derived from organic acids such as p-toluenesulfonic acid, salicylic acid, methanesulfonic acid, oxalic acid, succinic acid, citric acid, malic acid, lactic acid, fumaric acid, and the like. Example base-addition salts include those derived from ammonium, potassium, sodium and, quaternary ammonium hydroxides, such as for example, tetramethylammonium hydroxide. Chemical modification of a pharmaceutical compound into a salt is a known technique to obtain improved physical and chemical stability, hygroscopicity, flowability and solubility of compounds. See, e.g., H. Ansel et. al., Pharmaceutical Dosage Forms and Drug Delivery Systems (6th Ed. 1995) at pp. 196 and 1456-1457.

The pharmaceutical compositions comprise the compounds in a pharmaceutically acceptable carrier. A pharmaceutically acceptable carrier refers to sterile aqueous or nonaqueous solutions, dispersions, suspensions or emulsions, as well as sterile powders for reconstitution into sterile injectable solutions or dispersions just prior to use. Examples of suitable aqueous and nonaqueous carriers, diluents, solvents or vehicles include water, ethanol, polyols (such as glycerol, propylene glycol, polyethylene glycol and the like), carboxymethylcellulose and suitable mixtures thereof, vegetable oils (such as olive oil) and injectable organic esters such as ethyl oleate. The compounds can be formulated with pharmaceutically acceptable carriers or diluents as well as any other known adjuvants and excipients in accordance with conventional techniques such as those disclosed in Remington: The Science and Practice of Pharmacy, 19th Edition, Gennaro, Ed., Mack Publishing Co., Easton, Pa., 1995.

In various aspects, the disclosed pharmaceutical compositions comprise the disclosed compounds (including pharmaceutically acceptable salt(s) thereof) as an active ingredient, a pharmaceutically acceptable carrier, and, optionally, other therapeutic ingredients or adjuvants. The instant compositions include those suitable for oral, rectal, topical, and parenteral (including subcutaneous, intramuscular, and intravenous) administration, although the most suitable route in any given case will depend on the particular host, and nature and severity of the conditions for which the active ingredient is being administered. The pharmaceutical compositions can be conveniently presented in unit dosage form and prepared by any of the methods well known in the art of pharmacy.

Pharmaceutical compositions of the present invention suitable for parenteral administration can be prepared as solutions or suspensions of the active compounds in water. A suitable surfactant can be included such as, for example, hydroxypropylcellulose. Dispersions can also be prepared in glycerol, liquid polyethylene glycols, and mixtures thereof in oils. Further, a preservative can be included to prevent the detrimental growth of microorganisms.

Pharmaceutical compositions of the present invention suitable for injectable use include sterile aqueous solutions or dispersions. Furthermore, the compositions can be in the form of sterile powders for the extemporaneous preparation of such sterile injectable solutions or dispersions. In all cases, the final injectable form must be sterile and must be effectively fluid for easy syringability. The pharmaceutical compositions must be stable under the conditions of manufacture and storage; thus, preferably should be preserved against the contaminating action of microorganisms such as bacteria and fungi. The carrier can be a solvent or dispersion medium containing, for example, water, ethanol, polyol (e.g., glycerol, propylene glycol and liquid polyethylene glycol), vegetable oils, and suitable mixtures thereof.

Pharmaceutical compositions of the present invention can be in a form suitable for topical use such as, for example, an aerosol, cream, ointment, lotion, dusting powder, mouth washes, gargles, and the like. Further, the compositions can be in a form suitable for use in transdermal devices. These formulations can be prepared, utilizing a compound of the invention, or pharmaceutically acceptable salts thereof, via conventional processing methods. As an example, a cream or ointment is prepared by mixing hydrophilic material and water, together with about 5 wt % to about 10 wt % of the compound, to produce a cream or ointment having a desired consistency.

Pharmaceutical compositions of this invention can be in a form suitable for rectal administration wherein the carrier is a solid. It is preferable that the mixture forms unit dose suppositories. Suitable carriers include cocoa butter and other materials commonly used in the art. The suppositories can be conveniently formed by first admixing the composition with the softened or melted carrier(s) followed by chilling and shaping in molds.

In various aspects, the pharmaceutical compositions of this invention can include a pharmaceutically acceptable carrier and a compound or a pharmaceutically acceptable salt of the compounds of the invention. The compounds of the invention, or pharmaceutically acceptable salts thereof, can also be included in pharmaceutical compositions in combination with one or more other therapeutically active compounds.

The pharmaceutical carrier employed can be, for example, a solid, liquid, or gas. Examples of solid carriers include lactose, terra alba, sucrose, talc, gelatin, agar, pectin, acacia, magnesium stearate, and stearic acid. Examples of liquid carriers are sugar syrup, peanut oil, olive oil, and water. Examples of gaseous carriers include carbon dioxide and nitrogen.

In preparing the compositions for oral dosage form, any convenient pharmaceutical media can be employed. For example, water, glycols, oils, alcohols, flavoring agents, preservatives, coloring agents and the like can be used to form oral liquid preparations such as suspensions, elixirs and solutions; while carriers such as starches, sugars, microcrystalline cellulose, diluents, granulating agents, lubricants, binders, disintegrating agents, and the like can be used to form oral solid preparations such as powders, capsules and tablets. Because of their ease of administration, tablets and capsules are the preferred oral dosage units whereby solid pharmaceutical carriers are employed. Optionally, tablets can be coated by standard aqueous or nonaqueous techniques

A tablet containing the composition of this invention can be prepared by compression or molding, optionally with one or more accessory ingredients or adjuvants. Compressed tablets can be prepared by compressing, in a suitable machine, the active ingredient in a free-flowing form such as powder or granules, optionally mixed with a binder, lubricant, inert diluent, surface active or dispersing agent. Molded tablets can be made by molding in a suitable machine, a mixture of the powdered compound moistened with an inert liquid diluent.

In addition to the aforementioned carrier ingredients, the pharmaceutical formulations described above can include, as appropriate, one or more additional carrier ingredients such as diluents, buffers, flavoring agents, binders, surface-active agents, thickeners, lubricants, preservatives (including anti-oxidants) and the like. Furthermore, other adjuvants can be included to render the formulation isotonic with the blood of the intended recipient. Compositions containing a compound of the invention, and/or pharmaceutically acceptable salts thereof, can also be prepared in powder or liquid concentrate form.

In a further aspect, the effective amount is a therapeutically effective amount. In a still further aspect, the effective amount is a prophylactically effective amount.

In a further aspect, the effective amount is an amount of from about 0.1 mg/kg to about 0.4 mg/kg, about 0.1 mg/kg to about 0.3 mg/kg, about 0.1 mg/kg to about 0.2 mg/kg, about 0.2 mg/kg to about 0.4 mg/kg, about 0.3 mg/kg to about 0.4 mg/kg, or about 0.2 mg/kg to about 0.3 mg/kg. In a still further aspect, the effective amount is an amount of about 0.1 mg/kg, about 0.15 mg/kg, about 0.2 mg/kg, about 0.25 mg/kg, about 0.3 mg/kg, about 0.35 mg/kg, or about 0.4 mg/kg. In yet a further aspect, the effective amount is an amount of about 0.25 mg/kg.

In a further aspect, the composition comprises an effective amount of the agent known to treat spasticity. Examples of agents known to treat spasticity include, but are not limited to, baclofen, tizanidine, dantrolene sodium, diazepam, clonazepam, and gabapentin.

In a further aspect, the composition comprises an effective amount of an agent known to treat pain. Examples of agents known to treat pain include, but are not limited to, a nonsteroidal anti-inflammatory drug (NSAID) (e.g., flurbiprofen, ketorolac, ketoprofen, tolmetin, aspirin, ibuprofen, naproxen, indomethacin, sulindac, piroxicam, mefenamic acid, meloxicam, diclofenac, celecoxib, etodolac, etoricoxib, lumiracoxib, rofecoxib), an antimigraine agent (e.g., almotriptan, dihydroergotamine, eletriptan, ergotamine, frovatriptan, naratriptan, rizatriptan, sumatriptan, zomitriptan), a COX-2 inhibitor (e.g., celecoxib, rofecoxib, valdecoxib), acetaminophen, ziconotide, a narcotic (e.g., alfentanil, buprenorphine, butorphano, codeine, fentanyl, hydrocodone, hydromorphone, levorphanol, meperidine, methadone, morphine, nalbuphine, oxycodone, oxymorphone, propoxyphene, tramadol, tapentadol), and a salicylate (e.g., aspirin, diflunisal, magnesium salicylate, salsalate). In a still further aspect, the compound and the agent are administered sequentially. In yet a further aspect, pain is acute pain, chronic pain, or neuropathic pain. In an even further aspect, pain is neuropathic pain.

In a further aspect, the composition comprises an effective amount of a chemotherapeutic agent. Examples of chemotherapeutic agents include, but are not limited to, an alkylating agent (e.g., carboplatin, cisplatin, cyclophosphamide, chlorambucil, melphalan, carmustine, busulfan, lomustine, dacarbazine, oxaliplatin, ifosfamide, mechlorethamine, temozolomide, thiotepa, bendamustine, streptozocin), an antimetabolite agent (e.g., gemcitabine, 5-fluorouracil, capecitabine, hydroxyurea, mercaptopurine, pemetrexed, fludarabine, nelarabine, cladribine, clofarabine, cytarabine, decitabine, pralatrexate, floxuridine, methotrexate, thioguanine), an antineoplastic antibiotic agent (e.g., doxorubicin, mitoxantrone, bleomycin, daunorubicin, dactinomycin, epirubicin, idarubicin, plicamycin, mitomycin, pentostatin, valrubicin), a mitotic inhibitor agent (e.g., irinotecan, topotecan, rubitecan, cabazitaxel, docetaxel, paclitaxel, etopside, vincristine, ixabepilone, vinorelbine, vinblastine, teniposide), and a mTor inhibitor agent (e.g., everolimus, siroliumus, temsirolimus).

It is understood that the disclosed compositions can be prepared from the disclosed compounds. It is also understood that the disclosed compositions can be employed in the disclosed methods of using.

### E. KITS

In one aspect, disclosed are kits comprising an effective amount of a compound of a compound having a structure:

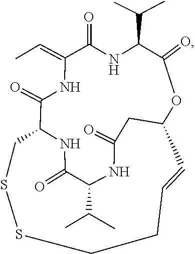 [SMILES: C/C=C1\NC(=O)[C@H]2CSSCC/C=C/[C@H](CC(=O)N[C@H](C(C)C)C(=O)N2)OC(=O)[C@H](C(C)C)NC1=O]

or a pharmaceutically acceptable salt thereof, and one or more of: (a) an agent known to treat spasticity; (b) an agent known to treat pain; (c) a chemotherapeutic agent; (d) instructions for treating spasticity; (e) instructions for treating pain; (f) instructions for administering the compound in connection with treating spasticity; and (g) instructions for administering the compound in connection with treating pain.

In a further aspect, pain is acute pain, chronic pain, or neuropathic pain. In a still further aspect, pain is neuropathic pain, for example, neuropathic pain caused by a peripheral nerve injury.

In a further aspect, the peripheral nerve injury is due to an injury. In a still further aspect, the injury is amputation or is due to surgical complication or trauma.

In a further aspect, the peripheral nerve injury is due to a disease. In a still further aspect, the disease is a metabolic disease. Examples of metabolic diseases include, but are not limited to, heart disease, diabetes, stroke, multiple sclerosis (MS) a lysosomal storage disease (e.g., Hurler syndrome, Gaucher disease, Niemann-Pick disease, Fabry disease, Tay-Sachs disease, a mucopolysaccharidoses (MPS) disease, Pompe disease), maple syrup urine disease, a glycogen storage disease (e.g., Von Gierke disease, Pompe's disease, Cori's disease. Andersen's disease, McArdle's disease, Hers™ disease, Tarui's disease), mitochondrial disease (e.g., mitochondrial myopathy, diabetes mellitus and deafness (DAD), Leber's hereditary optic neuropathy (LHON), Leigh syndrome, neuropathy, ataxia, retinitis pigmentosa, and ptosis (NARP), myoneurogenic gastrointestinal encephalopathy (MNGIE), MERRF syndrome, MELAS syndrome, mitochondrial DNA depletion syndrome), a peroxisomal disorder (e.g., X-linked adrenoleukodystrophy (X-ALD), Zellweger syndrome (ZS), neonatal adrenoleukodystrophy (NALD), infantile Refsum disease (IRD), rhizomelic chondrodysplasia punctata (RCDP), Zellweger-like syndrome), and a metal metabolism disorder (e.g., hypermanganesemia with dystonia 1, hypermanganesemia with dystonia 2, Wilson disease, acaeruloplasminaemia, cerebellar ataxia, hypoceruloplasminemia, neuroferritinopathy, hyperferritinemia-cataract syndrome, L-ferritin deficiency, spastic paraplegia, HARP syndrome, pontocerebellar hypoplasia, infantile neuroaxonal dystrophy, Parkinson's disease, Woohouse-Sakati syndrome, Kufor-Rakeb syndrome, hemochromatosis). In yet a further aspect, the metabolic disease is selected from diabetes and multiple sclerosis (MS).

In a further aspect, the kit comprises the agent known to treat spasticity. Examples of agents known to treat spasticity include, but are not limited to, baclofen, tizanidine, dantrolene sodium, diazepam, clonazepam, and gabapentin. In a still further aspect, the compound and the agent known to treat spasticity are co-packaged. In yet a further aspect, the compound and the agent known to treat spasticity are co-formulated.

In a further aspect, the kit comprises the agent known to treat pain. Examples of agents known to treat pain include, but are not limited to, a nonsteroidal anti-inflammatory drug (NSAID) (e.g., flurbiprofen, ketorolac, ketoprofen, tolmetin, aspirin, ibuprofen, naproxen, indomethacin, sulindac, piroxicam, mefenamic acid, meloxicam, diclofenac, celecoxib, etodolac, etoricoxib, lumiracoxib, rofecoxib), an antimigraine agent (e.g., almotriptan, dihydroergotamine, eletriptan, ergotamine, frovatriptan, naratriptan, rizatriptan, sumatriptan, zomitriptan), a COX-2 inhibitor (e.g., celecoxib, rofecoxib, valdecoxib), acetaminophen, ziconotide, a narcotic (e.g., alfentanil, buprenorphine, butorphano, codeine, fentanyl, hydrocodone, hydromorphone, levorphanol, meperidine, methadone, morphine, nalbuphine, oxycodone, oxymorphone, propoxyphene, tramadol, tapentadol), and a salicylate (e.g., aspirin, diflunisal, magnesium salicylate, salsalate). In a still further aspect, the compound and the agent are administered sequentially. In yet a further aspect, pain is acute pain, chronic pain, or neuropathic pain. In an even further aspect, pain is neuropathic pain. In a still further aspect, the compound and the agent known to treat pain are co-packaged. In yet a further aspect, the compound and the agent known to treat pain are co-formulated.

In a further aspect, the kit comprises the chemotherapeutic agent. Examples of chemotherapeutic agents include, but are not limited to, an alkylating agent (e.g., carboplatin, cisplatin, cyclophosphamide, chlorambucil, melphalan, carmustine, busulfan, lomustine, dacarbazine, oxaliplatin, ifosfamide, mechlorethamine, temozolomide, thiotepa, bendamustine, streptozocin), an antimetabolite agent (e.g., gemcitabine, 5-fluorouracil, capecitabine, hydroxyurea, mercaptopurine, pemetrexed, fludarabine, nelarabine, cladribine, clofarabine, cytarabine, decitabine, pralatrexate, floxuridine, methotrexate, thioguanine), an antineoplastic antibiotic agent (e.g., doxorubicin, mitoxantrone, bleomycin, daunorubicin, dactinomycin, epirubicin, idarubicin, plicamycin, mitomycin, pentostatin, valrubicin), a mitotic inhibitor agent (e.g., irinotecan, topotecan, rubitecan, cabazitaxel, docetaxel, paclitaxel, etopside, vincristine, ixabepilone, vinorelbine, vinblastine, teniposide), and a mTor inhibitor agent (e.g., everolimus, siroliumus, temsirolimus). In a still further aspect, the compound and the chemotherapeutic agent are co-packaged. In yet a further aspect, the compound and the chemotherapeutic agent are co-formulated.

It is understood that the disclosed kits can be prepared from the disclosed compounds, products, and pharmaceutical compositions. It is also understood that the disclosed kits can be employed in connection with the disclosed methods of using.

### F. EXAMPLES

The following examples are put forth so as to provide those of ordinary skill in the art with a complete disclosure and description of how the compounds, compositions, articles, devices and/or methods claimed herein are made and evaluated, and are intended to be purely exemplary of the invention and are not intended to limit the scope of what the inventors regard as their invention. Efforts have been made to ensure accuracy with respect to numbers (e.g., amounts, temperature, etc.), but some errors and deviations should be accounted for. Unless indicated otherwise, parts are parts by weight, temperature is in ° C., or is at ambient temperature, and pressure is at or near atmospheric.

The Examples are provided herein to illustrate the invention, and should not be construed as limiting the invention in any way. Examples are provided herein to illustrate the invention and should not be construed as limiting the invention in any way.

### 1. Preliminary Studies Involving the Use of Romidepsin to Treat Spasticity

a. Rac1 Regulated Dendritic Spine Dysgenesis on α-Motor Neurons Contribute to Spasticity after SCI

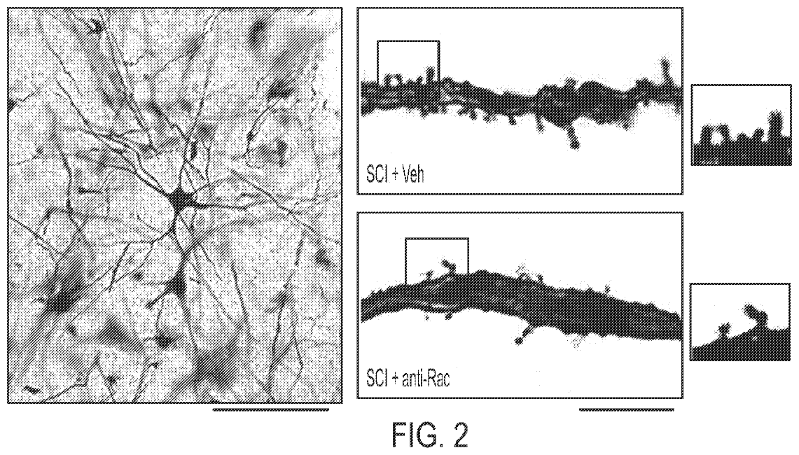

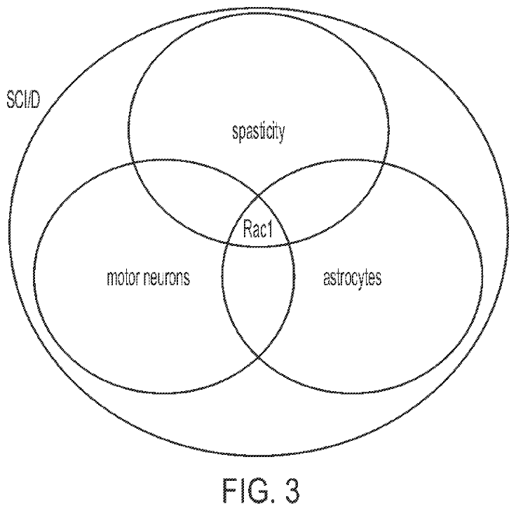

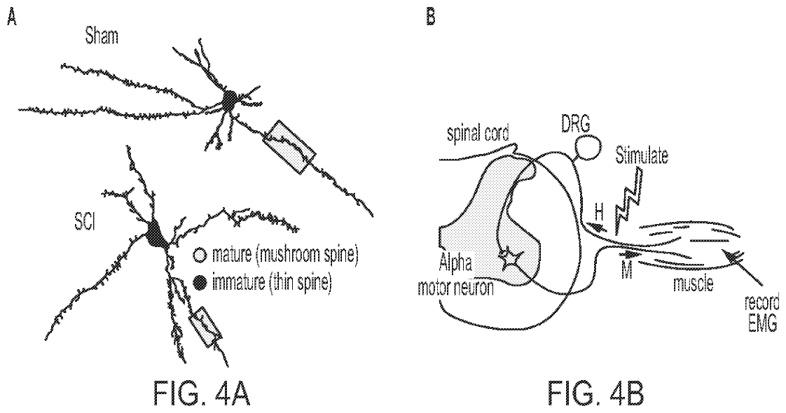

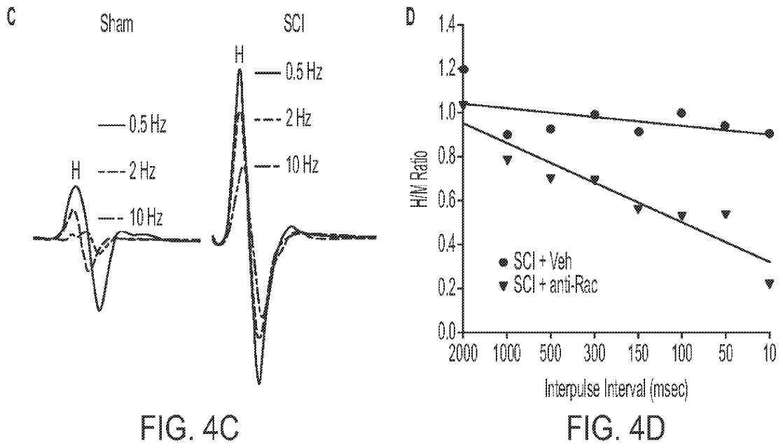

A published longitudinal study demonstrated that Rac1 activity is necessary and sufficient for dendritic spine dysgenesis in α-motor neurons and spasticity after SCI (Bandaru et al., 2015) (FIG. 2 and FIG. 4A-D).

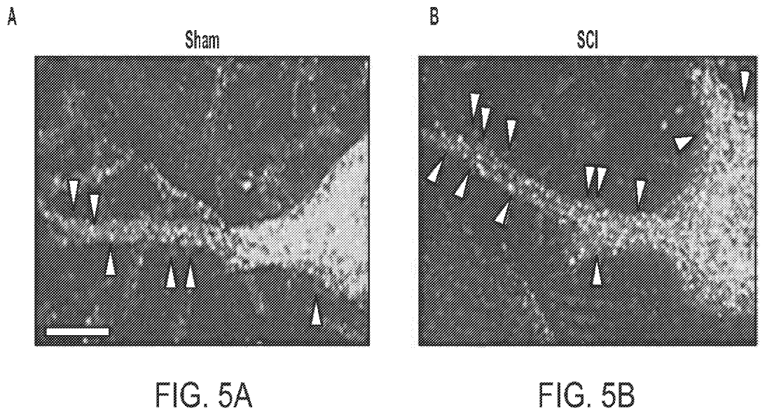

Four-weeks after SCI, a loss of rate-dependent-depression (RDD) of the H-reflex (i.e., a clinical sign of spasticity) was observed. SCI also increased the number of vesicular glutamate transporter-1 (VGLUT1)-positive boutons contacting α-motor neurons labeled with cholera toxin-B (CTB) (FIG. 5A and FIG. 5B), demonstrating an increase in excitatory presynaptic innervation on α-motor neurons. These outcomes were partly reversible with an intrathecal Rac1-inhibitor treatment. Notably, spasticity and cellular changes relapsed within 3-days after drug withdrawal.

b. AAV-Mediated Cre-Lox Conditional Rac1 Knockout in α-Motor Neurons Attenuates Spasticity

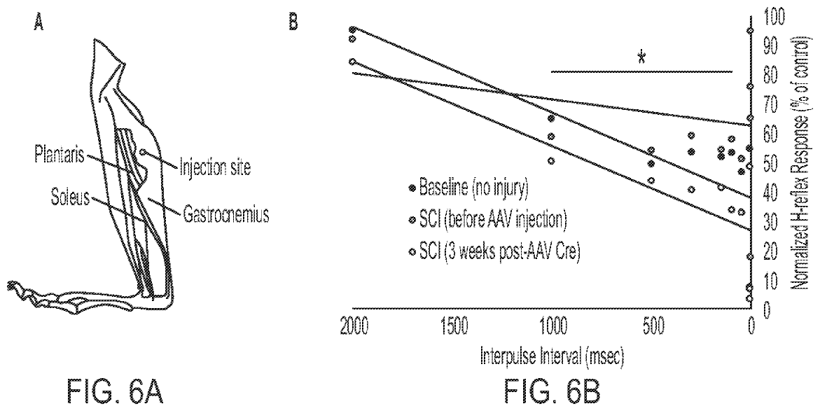

To validate a genetic approach to selectively knockout Rac1 in α-motor neurons, AAV2 carrying Cre-expressing constructs were injected into hind limb muscle tissue of transgenic mice (Rac1 “floxed” mice with tomato reporter; Rac1^{f/ft/t}) (FIG. 6A). The evoked H-reflex response was measured using percutaneous electrodes before/after SCI (contusion injury, Infinite Horizon Impactor; 50 kDyN force; spinal segment ˜T11), and before/after AAV-Cre injections (FIG. 6B). Before AAV injection, SCI animals exhibited reduced RDD; whereas in the same animals 3-weeks after AAV injection, RDD returned close to baseline control levels.

c. Spinal Cord Tissue “Clearing” to Analyze Dendritic Spines and Other Anatomical Correlates

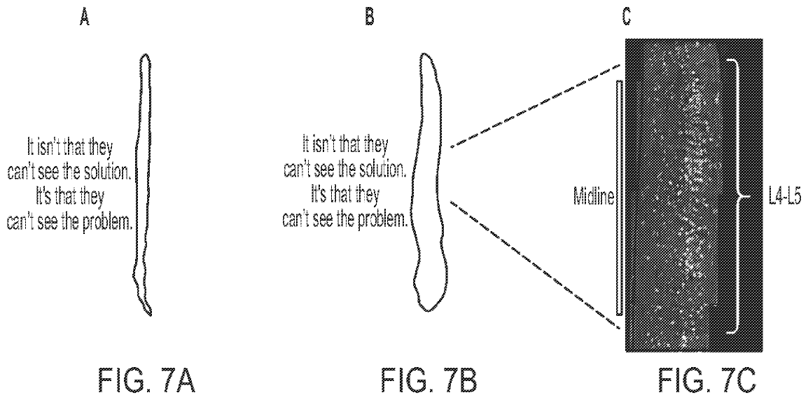

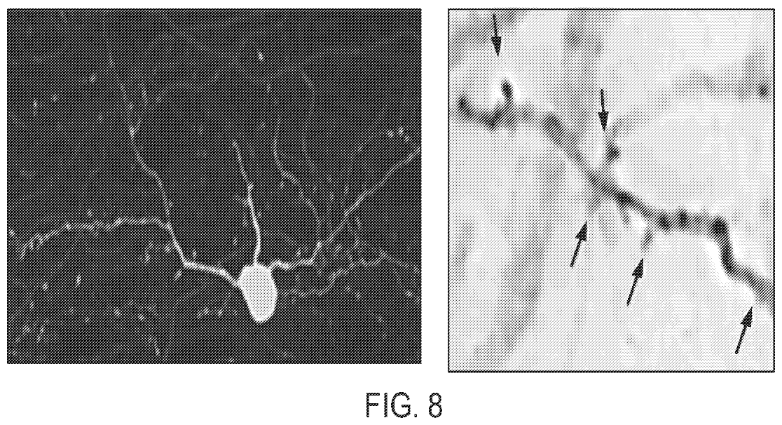

Previous studies have used Golgi-staining protocols to visualize and analyze cellular morphology (Bandaru et al., 2015; Zhao et al., 2016). To image an entire motor neuron's structure following viral infection and reporter expression, a routine whole-tissue clearing method was established that permits imaging without the need for thin-tissue sectioning (i.e., SWITCH protocol) (Murray et al., 2015) (FIG. 7A-C). This method also allows for multi-antigen staining for co-labeling studies. As shown in FIG. 7C, it was demonstrate that cleared spinal cord tissue permits visualization of infected motor neurons in the ventral horn. Here, a reliable infection rate of >50-80% of ventral α-motor neurons was observed in Rexed lamina IX (using stereotaxic coordinates) within the L4-L5 spinal segments. Alpha-motor neurons were identified based on published morphological criteria (Benson et al., 2017; Hashizume et al., 1988; Tan et al., 2012a). Non-neuronal and muscle tissue remained uninfected, based partly on co-localization studies with GFAP and Iba1 (data not shown). Note that it is also possible to visualize and analyze dendritic spines of infected motor neurons in cleared tissue (FIG. 8).

d. Disruption of SCI-Induced Dendritic Spine Dysgenesis Through Rac1 Knockout in Astrocytes

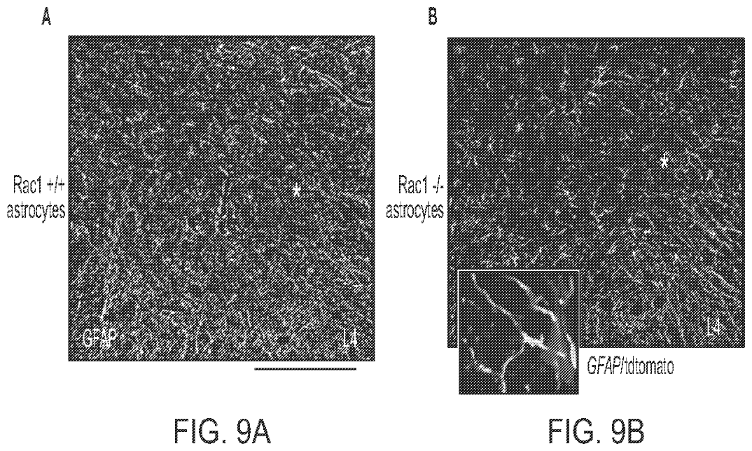

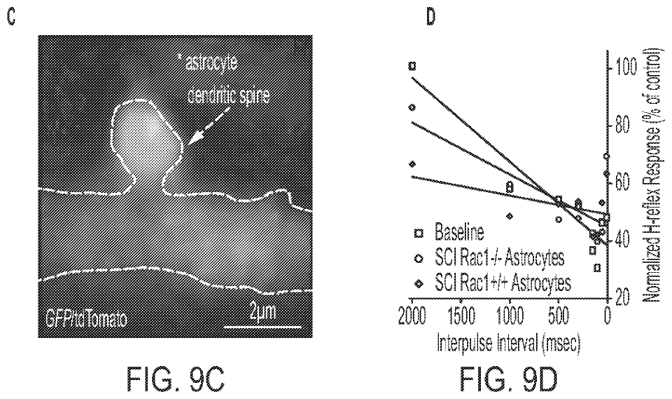

A viable colony of transgenic mice lacking astrocytic Rac1 (GFAP promoter driven Cre-expression in Rac1 “floxed” mice with tomato reporter) was established (FIG. 9A-D). These transgenic animals behave normally into adulthood, without observable behavioral deficits (Senger et al., 2002). In pilot SCI studies, a qualitative lack of SCI-induced astrogliosis was observed in astrocyte Rac1 knockout animals (FIG. 9A and FIG. 9B). To ensure equivalent labeling of astrocytes in histological analyses, all tissues will be immunostained for GFAP, which will co-localize with tdTomato reporter (red) (FIG. 9B, inset). To investigate a putative interaction between astrocytes and motor neurons, it was demonstrated that virally-infected α-motor neurons express GFP reporter with visible dendritic spines, which are enveloped with astrocytic processes (expressing red tdTomato reporter; z-stack image) (FIG. 9C) (Perez-Alvarez et al., 2014). A possible difference in evoked H-reflex excitability was also observed in astrocyte Rac1 knockout animals following SCI, as compared with baseline or control SCI animals (FIG. 9D).

e. Validation of Shrna Knockdown Constructs Toward Targeting Rac1

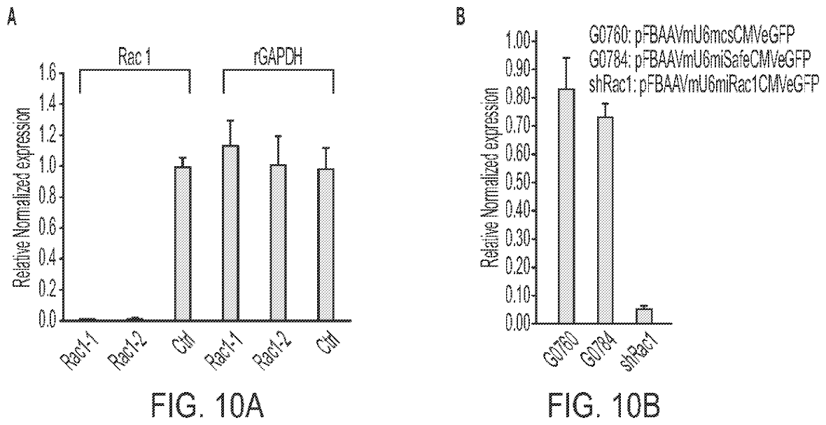

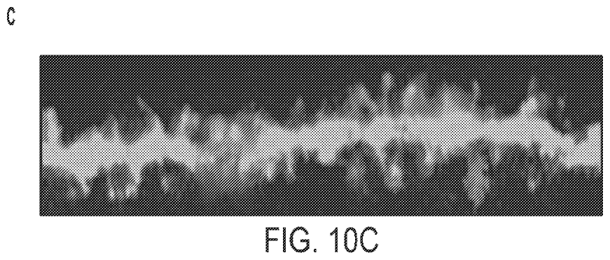

The utility of a gene therapy “platform” targeting Rac1 to reduce spasticity will be investigated, as detailed further herein. To validate the custom constructs (Gene Transfer Vector Core; Iowa University), a neuronal cell line (ND7/23) was transfected with the naked siRNA constructs (FIG. 10A) and AAV-shRNA constructs (FIG. 10B). In both testing paradigms, Rac1 mRNA expression decreased without pleotropic effects on other closely related proteins. Other Rac isoforms, Rac2 or Rac3, are not expressed in neurons and do not effect dendritic spine morphologies (Impey et al., 2010). Additional validation studies, e.g., histological/Rac1 pull-down assays, will be performed in future studies, as detailed elsewhere herein. Intramuscular injections of AAV2 with custom constructs are successful; infecting spinal motor neurons and expressing sufficient green fluorescent protein (GFP) for dendritic spine analyses (FIG. 10C).

f. PAK1 Inhibition Disrupts Dendritic Spine Dysgenesis

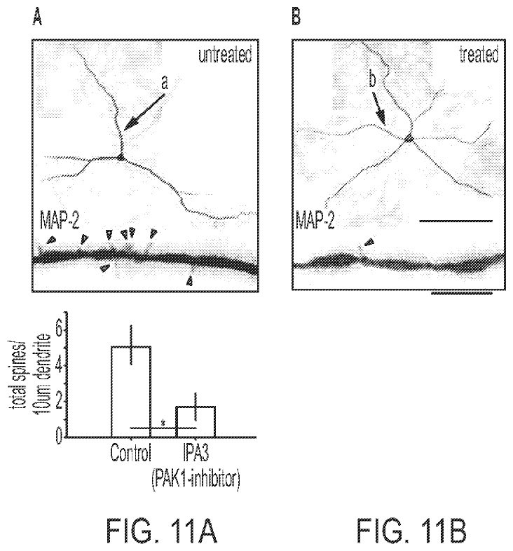

To assess whether PAK1 inhibition would disrupt dendritic spines, a morphological correlate of spasticity, the effect of a non-pharmaceutical grade PAK1 inhibitor, IPA3, on primary cultured spinal cord dorsal horn neurons was measured using previous methods (Tan et al., 2011). As shown in FIG. 11, spine density decreases with PAK1 inhibitor treatment.

g. Romidepsin Bioavailability in the Spinal Cord, Drug-Tissue Action on Neurons, and Functional Efficacy

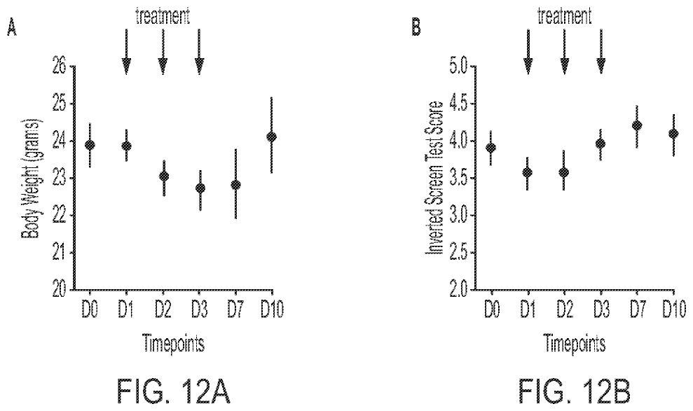

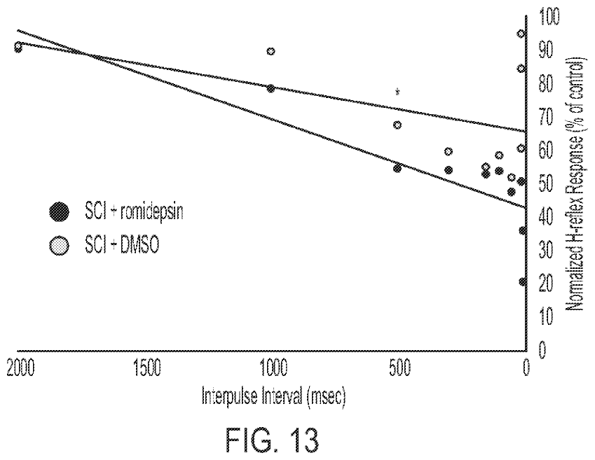

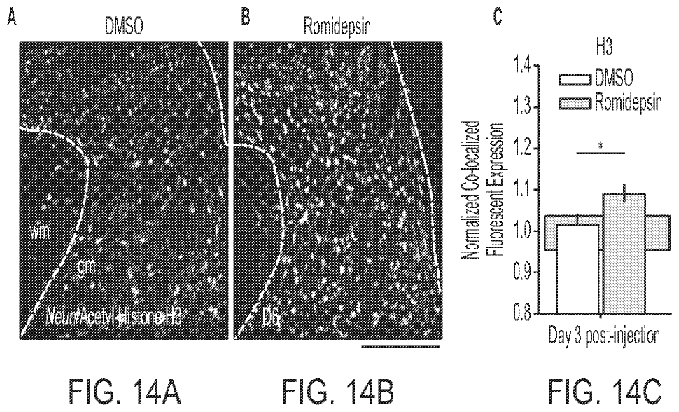

As detailed elsewhere herein, the feasibility of “repurposing” a clinically approved drug to address spasticity in SCI will be evaluated. Here, testing the efficacy of romidepsin, an FDA-approved drug, to reduce PAK1 activity in the spinal cord and SCI-induced spasticity has begun. The maximum tolerated dose (MTD) for romidepsin has been established in a SCI mouse model (using endpoint criteria of weight loss and drug-induced locomotor deficit, e.g., paw grip-strength, mobility, as compared with vehicle) (romidepsin MTD=once/day IP injection over 3 days; 0.1 ml, 1.25 mg/kg/injection) (FIG. 12A and FIG. 12B). Initial test dosages were calculated from human trial data using FDA guidelines for converting drug dosage from human to animal. Preliminary EMG studies show that romidepsin treatment in SCI animals partially restored RDD, i.e., reduced spasticity, as compared with vehicle control (FIG. 13). Immunohistology shows that romidepsin has bioavailability in the spinal cord parenchyma, as shown using drug response-biomarker expression, e.g., histone acetylation (FIG. 14A-C) (VanderMolen et al., 2011).

### 2. Evaluation of Romidepsin Efficacy in a SCI Spasticity Model

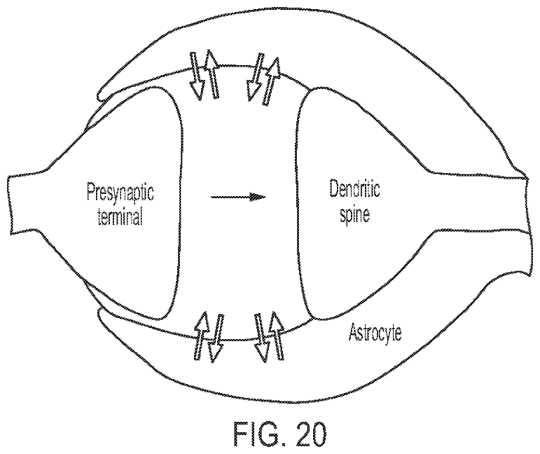

Studies have established Rac1 as a potential target for the treatment of spasticity following SCI, however, the involvement of Rac1 in multiple cellular pathways diminishes its utility as a target for clinical drug development. The National Institutes of Health (NIH) has advocated for the identification of clinically approved drugs that can be “repurposed” expeditiously to treat other diseases (Brooks et al., 2014; Brooks et al., 2016). To leverage this strategy, PAK1 (P21 (RAC1) Activated Kinase-1) has been identified, which has already been used as a “druggable” target for cancer and neurological disease in humans (Bertino et al., 2011; Kichina et al., 2010; Nikolic, 2008) (FIG. 20). Importantly, PAK1 is a downstream effector linking Rac1 to dendritic spine reorganization and is required for dendritic spine dysgenesis associated with several neuropsychiatric disorders (Baker-Herman et al., 2004; Boda et al., 2008; Hayashi et al., 2007; Liu et al., 2009; Ma et al., 2012).

Romidepsin (aka FK228) is a potent high-affinity HDAC inhibitor (Hayashi et al., 2007). Bioavailable concentrations of romidepsin at 0.1-1 nM significantly reduces PAK1 kinase activity without changing PAK1 protein level (Hirokawa et al., 2005; Maruta, 2011). The drug's active metabolites can passively penetrate through the blood brain barrier (BBB) in relatively low concentrations when administered systemically in non-human primates or rodents (Berg et al., 2004) (see FIG. 14A-C). Much higher CNS bioavailability is possible after SCI or intrathecal delivery (Matsushita et al., 2015). Here, the utility of “repurposing” romidepsin, a clinically available drug to disrupt PAK1, to relieve spasticity after SCI was evaluated.

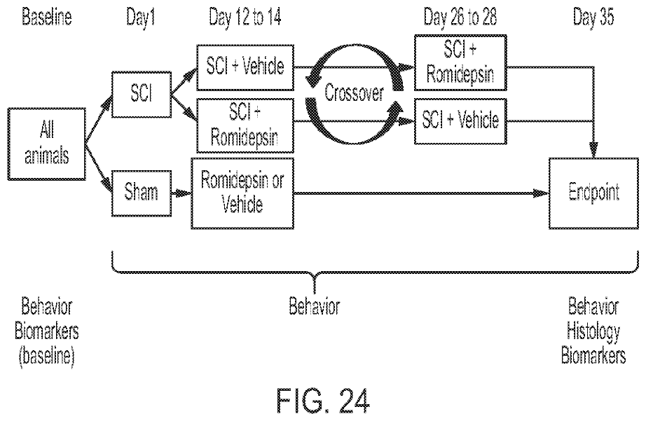

Study Design: Four animal groups were prepared (adult m/f mix; 6-8 weeks old; n=60/group); 1) Sham+DMSO/vehicle, 2) Sham+Romidepsin, 3) SCI+DMSO/vehicle, and 4) SCI+Romidepsin. All animals in this study were reporter mice expressing fluorescent GFP driven by the neuron-specific thymus cell antigen-1 promotor (Thy1-GFP). Thy1-GFP mice are useful because of the reporter's specificity for neurons, clarity of dendritic spines in vivo, and are an available commercial stock from Jackson Laboratories. Romidepsin at MTD or DMSO vehicle was administered as three intrathecal (i.t.) injections two-weeks after SCI or Sham surgeries. The experiment was performed as a “vehicle-controlled crossover study.” (see FIG. 24). This study design leverages the expected transient effect of romidepsin on spasticity due to the drug's relatively short half-life (<10 hours) based on published PK/PD studies (Berg et al., 2004). As such, this design space should reveal a close functional relationship between romidepsin treatment, and attenuated dendritic spine dysgenesis and spasticity.

The maximum tolerated dose (MTD) for romidepsin has been established as detailed above (once/day for 3 days; 10 μl per IP injection at 1.25 mg/kg; diluted in 1% DMSO; FIG. 12A-B and FIG. 13). The initial dosages in the pilot dose-response study were calculated using FDA guidelines for converting drug dosage between species (animal mg/kg dose×animal km=human mg/kg dose×human km) from the human MTD for lymphoma treatment (FDA website, last accessed on Nov. 21, 2017). Vehicle (1% DMSO) was used as a control treatment.

Outcome Measures: On days 14, 28, and 35 post-SCI, spinal cord and brain tissue were collected for histological analyses. Tissue was processed using SWITCH clearing and immunohistochemistry performed to analyze protein expression levels of inflammatory markers, i.e., GFAP/OX42 for astro/microgliosis, cfos, p38, p-Raf, or PAK1 activity (Stamboulian et al., 2010); Tan et al., 2011; Tan et al., 2008). Because mice are Thy1-GFP transgenic animals, α-motor neurons were identified using established morphological criteria, including soma size and topographical location (Bandaru et al., 2015; Tan et al., 2012a). GFP-expressing dendritic spines were visualized, and their morphology profiled in association with spasticity. All data was statistically compared across groups (Bandaru et al., 2015; Zhao et al., 2016). To determine the efficacy of romidepsin treatments on SCI-induced spasticity, anatomical and biochemical data were correlated with spasticity outcomes.

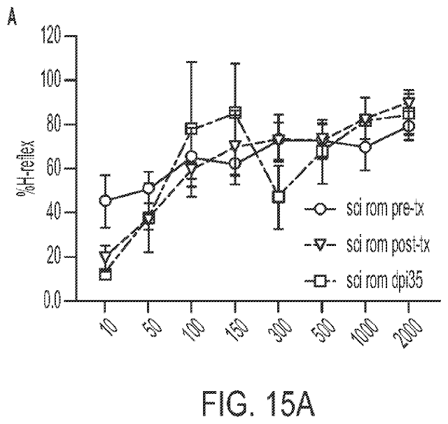

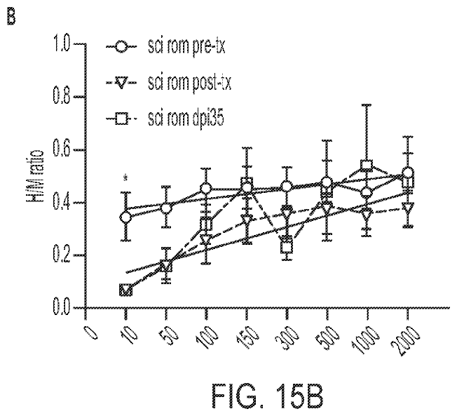

Referring to FIG. 15A and FIG. 15B, male and female adult mice underwent surgeries to produce a clinically-relevant SCI and spasticity condition. Animals were administered romidepsin through IP injection at 0.25 mg/kg or control vehicle (n=10/group). Early data shows significant restoration of RDD following SCI with romidepsin treatment (see H/M ratio; FIG. 15B). Without wishing to be bound by theory, this indicates that the ability of motor neurons to fire and communicate with muscles is improved after romidepsin administration, as compared to control.

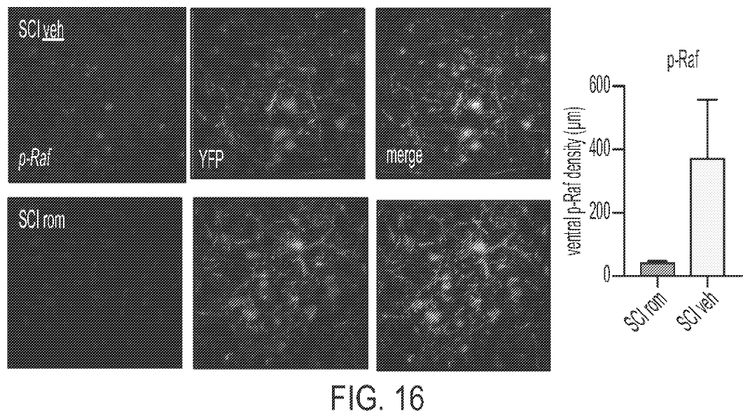

To confirm bioavailability of romidepsin within the spinal cord parenchyma tissue, histological analysis was performed using known biomarkers of drug-tissue response. As shown in FIG. 16, romidepsin exposure significantly decreases the expression of p-Raf on Thy-YFP expressing cells (which are neurons). Without wishing to be bound by theory, this indicates that romidepsin is able to penetrate the injured spinal cord and reach its target site of action on neurons within the ventral spinal cord.

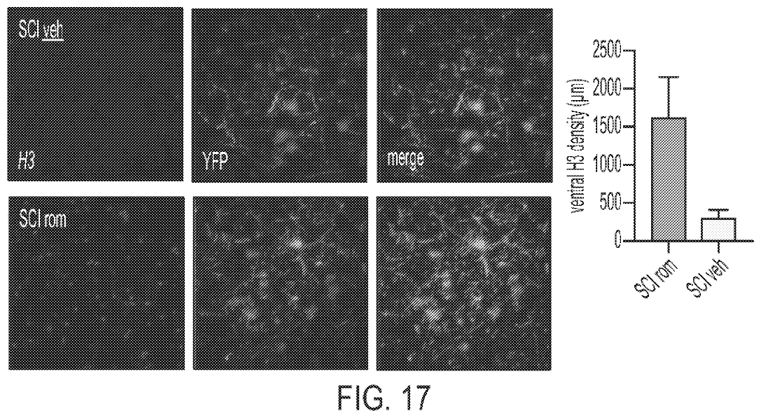

To monitor in vivo drug-response using established clinical biomarkers from all animals before and after treatment with romidepsin, histone acetylation was assessed in spinal cord tissue, peripheral blood mononuclear cells (PBMCs), and monocytes from CSF (i.e., ELISA pharmacodynamics assessment) (Cotto et al., 2010); VanderMolen et al., 2011). Liquid samples were collected using routine blood or spinal CSF draws. As shown in FIG. 17, romidepsin administration led to a significant increase in H3 density, which is a marker for de-acetylation within the tissues of the spinal cord, as compared with control treated animal tissues.

### 3. Design and Methods for Future Research Involving the Use of Romidepsin to Treat Spasticity

a. Research to Determine the Contribution of Rac1-Activity in α-Motor Neurons and Astrocytes in Spasticity after SCI
(i) to Determine the Effectiveness of Conditional Rac1 Knockout in Spinal Cord α-Motor Neurons to Relieve Dendritic Spine Dysgenesis and Spasticity after SCI

Rationale: Pharmaceuticals have dose-limiting side effects and non-specific tissue action, which confound mechanistic insight. To extend previous findings, a Cre-Lox system will be used to knockout Rac1 expression in spinal cord α-motor neurons. Without wishing to be bound by theory, it is expected that primary outcome data will reveal the contribution of motor neuron Rac1 signaling in spasticity after SCI.

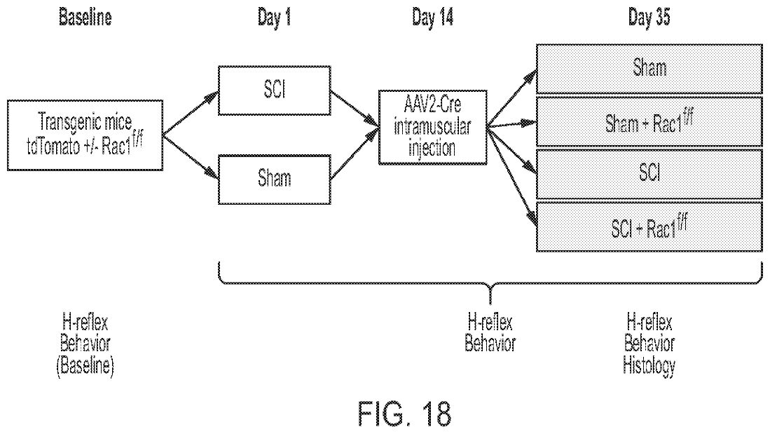

Study Design: Four animal groups will be prepared (male/female equal mix; 6-8 weeks old; n=30/group): 1) Sham, 2) Sham+Rac1^{f/f}, 3) SCI, and 4) SCI+Rac1^{f/f} (see Study Design in FIG. 18). All animals are transgenic tdTomato reporter (red) mice with or without loxp sites flanking the Rac1 gene (tdTomato+Rac1^{f/f}). All AAV2 vectors are commercially available (Gene Transfer Vector Core; Iowa University). Neuronal expression of Cre-recombinase following viral infection will knockout Rac1 expression (Rac1^{f/f} “floxed” dependent). Preliminary data demonstrates the utility of this approach (FIG. 6A-B and FIG. 8).

To induce spasticity, a mild contusion SCI will be performed using an Infinite Horizon (IH) impactor device (segmental level T11; 50 kDyn force) (preliminary data in FIG. 6A and FIG. 6B). To confirm SCI reproducibility, biomechanical data will be collected from the IH device, i.e., force and tissue displacement (Scheff et al., 2003). This SCI model can produce reliable and consistent signs and symptoms of clinical spasticity (Ahmed, 2014; Bandaru et al., 2015; Boulenguez et al., 2010; Carp et al., 2006).

Outcome Assessments: Outcome assessments will be performed by blinded investigators at three time points: at baseline, Day 14 post-SCI/Sham (before AAV injections), and Day 35. Before any injury, naïve animals will be tested for baseline function. This includes gross locomotor assessments using a CatWalk gait-analysis system (Noldus; Version 9.1) and the Basso Mouse Scale (BMS). Final spasticity and locomotor testing will be performed three-weeks after intramuscular AAV-Cre injection (Day 35 post-SCI). Immediately after final testing, animals will be euthanized and spinal cord tissue collected for biochemical and histological studies. To histologically monitor the extent of AAV infection, brain and dorsal root ganglia (L4-L5) will also be collected.

H-reflex electrophysiology-EMG recordings of evoked H-reflex will be performed in the plantar muscle group (Bandaru et al., 2015; Benson et al., 2017; Boulenguez et al., 2010; Nielsen et al., 2007). The plantar reflex has been shown to reflect similar changes in reflexes elicited in larger hind limb muscles, i.e., tibialis anterior, soleus, and gastrocnemius, which are innervated from α-motor neurons in spinal L4/L5 (Lee et al., 2009; Valero-Cabre et al., 2004). To perform longitudinal studies over time in the same animals, electrodes will be inserted percutaneously. To test the H-reflex, a paired-pulse stimulation paradigm will be applied: a control pulse and test stimulus (0.2 ms square) with a range of interpulse intervals (5-2000 ms). Three trials (10 sweeps/trial) will be recorded for each paired-pulse. Rectified traces will be analyzed. For comparisons, the peak amplitude of the H and M responses to the test pulse will be converted into a percentage (%) of the peak amplitude response to the control pulse (testᵣ/condᵣ×100). The H/M ratio will be calculated using the peak amplitude of M-wave and H-reflex following the test pulse. H-reflex EMG studies provide specific readout only of the monosynaptic circuit, and is therefore an accurate measure of the spinal stretch reflex response without the confounds of supraspinal, interneuronal, or other motor neuron sub-type input (Nielsen et al., 2007).

Behavioral studies—To complement electrophysiological testing, a blinded observer will assess behavioral spasticity events in a swimming-test designed for SCI models (Ryu et al., 2017). The test will be performed in a Plexiglas chamber filled with water (23° C.; 20 cm deep) with a submerged observation mirror. A trial consists of an animal swimming from one end of the chamber to a submerged platform at the other end. Five trials/animal will be performed at each experimental endpoint. The total number of spastic movements (defined as flexed trunk posture with extended or jerking hind limbs) over 5 trials will be measured from high-speed video recordings (>120 fps) (Ryu et al., 2017). The ipsilateral-injected side of animals will be scored alone or in combination with bilateral scores, and compared across groups. To measure gross locomotor function, the BMS test, an established open-field scoring paradigm for mice with SCI (i.e., a 9-point ordinal scale for locomotor function) (Basso et al., 2006), will be used. A CatWalk gait analysis system (Noldus) will also be used. Primary outcomes from the CatWalk will be stride length, paw position/coordination, and regularity index (e.g., deviations from standard gait pattern) (Hunanyan et al., 2013). Each group will undergo these tests 14 days after SCI. A day later (Day 15 post-SCI), 1-7×10¹³ particles of AAV2-Cre will be injected into the soleus muscle group of the left hind limb.

Biochemical molecular biology—To assess whether viral infection disrupts Rac1 expression, fresh spinal cord tissue will be collected from a subpopulation of animals at experimental endpoint (n=5/group at Day 35), and a Rac1-Pak1 pulldown assay performed, as described previously, and available as a commercial kit (Pierce, Rockford, IL) (Yang et al., 2006). Routine antibody/immunohistology will also be used to detect levels of Rac1 protein in chemically fixed lumbar enlargement (L4-L5) spinal cord tissue (n=10/group) (Corbetta et al., 2009).

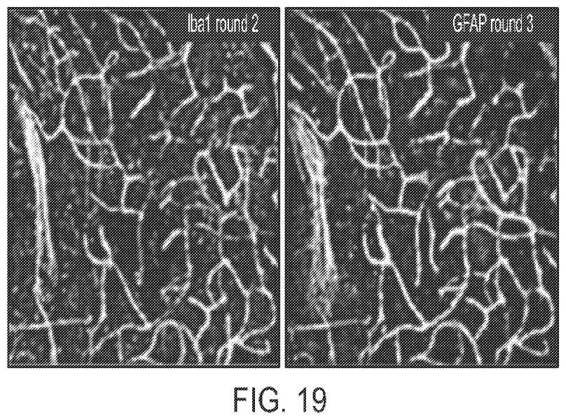

Anatomy histology Spinal cord tissue will be processed using the SWITCH tissue clearing protocol (Murray et al., 2015) (n=15/group) (FIG. 7A-C). To quantify viral infection efficiency, the number of motor neurons expressing tdTomato reporter will be compared to the total number of identified α-motor neurons (based on topographical location and co-localized ChaT-immunoreactivity) (Chakrabarty et al., 2009; Tan et al., 2012a). For dendritic spine analyses, confocal microscopy will be used to create z-stack 3D reconstructions of motor neurons and their dendritic spines. A blinded experimenter will analyze dendritic spine morphologies using previous digital reconstruction methods modified for fluorescent visualization (Benson et al., 2017; Kim et al., 2006). Neurolucida software (MBF Biosciences) will be used to profile dendritic spine density, distribution, and shape/volume. SWITCH clearing also permits post-mortem fluorescent antibody labeling of other antigens (FIG. 19). Spinal cord will be co-labeled with antibodies for motor neurons, e.g., ChAT, Rac1 (total protein: active/inactive kinase), Iba1 (microglia), GFAP (astrocytes), and other inflammatory markers. To quantify protein levels, a percent field analysis (i.e., proportional area of immunoreactivity of protein/total area measured) will be used, and identical background signal normalization and tissue/image processing will be ensured. Changes in presynaptic terminals will be assessed using immunoreactivity methods for VGluT1 puncta, which increase in density along motor neuron dendrites after SCI (Alvarez et al., 2004; Bandaru et al., 2015; Zhao et al., 2016) (FIG. 5A and FIG. 5B). All data will be compared across groups using appropriate parametric or non-parametric statistical models, e.g. ANOVA or Rank-sum tests, and corrected for repeated measure errors, e.g., Bonferroni method.

(ii) to Determine Whether Conditional Rac1 Knockout in Astrocytes can Reduce Spasticity after SCI

Rationale: Astrocyte processes are intimately associated with dendritic spines (FIG. 9C), which has a direct functional effect on neuronal transmission (Bourne et al., 2008; Brockett et al., 2015; Nishida et al., 2007; Perez-Alvarez et al., 2014) (FIG. 20). Astrocytes also contribute to maintaining neuronal hyperexcitability in the spinal cord after injury (Halassa et al., 2007; Perez-Alvarez et al., 2014; Scholz et al., 2007). Importantly, emerging evidence has demonstrated that hypertrophic or reactive astrocyte activity following injury is Rac1-dependent (Racchetti et al., 2012). Here, whether the absence of Rac1 expression in astrocytes reduces the presence of spasticity after SCI will be assessed.

Study Design: Two SCI animal groups (male/female equal mix; 6-8 weeks old; n=20/group): 1) SCI+GFAP-Cre/Rac1^{f/f}/tomato, and 2) SCI+GFAP-Cre/tomato will be prepared. The general study design, SCI model, and experimental endpoints are similar to that detailed above. A viable breeding colony of transgenic mice lacking astrocytic Rac1 expression (GFAP-Cre/Rac1f/f/tomato; GFAP promoter driven Cre-expression in Rac1 “floxed” mice with tomato reporter) has already been established (see FIG. 9A-D). These transgenic animals behave normally into adulthood, without observable behavioral deficits (Senger et al., 2002). TdTomato reporter (red) expresses only in astrocytes in these animals. For control animals, i.e., normal Rac1 expression, a GFAP-Cre/tomato transgenic mouse line, which expresses the tomato reporter in astrocytes, will be used. To ensure equivalent labeling of astrocytes in histological analyses, all tissues will be immunostained for GFAP using an appropriately colored fluorescent tag (FIG. 9B, inset). Routine mouse genotyping will be used along with histological methods to validate astrocytic Rac1 knockout. Fendritic spine profiling will also be employed to further understand the relationship between Rac1-knockout in astrocytes and motor neuron plasticity after SCI.

Outcome Measures: Functional/anatomical outcome assessments will be similar to those described above. To label α-motor neurons, AAV2-GFP will be intramuscularly injected into soleus muscle to deliver constructs for green fluorescent protein (GFP) reporter expression. The infection yield (>50%) of α-motor neurons using AAV2-GFP vectors has been validated. Dendritic spines are visible with this method (FIG. 10C). Astrocyte morphology will be compared between the two groups; without wishing to be bound by theory, Rac1 knockout is expected to reduce hypertrophic astrocyte morphology, i.e., swollen soma with increased thickness of their main processes.

To monitor other effects on the body, including inflammatory response, brain and DRG tissues will be collected. To analyze dendritic spine profiles, and other immuno-histological assessments, SWITCH clearing will be used on spinal cord samples. Outcome data will be statistically compared across groups using appropriate parametric or non-parametric statistical models, e.g. ANOVA or Rank-sum tests, and corrected for repeated measure errors post hoc, e.g., Bonferroni, Dunn's tests.

To describe a putative interaction between astrocytes and motor neurons (FIG. 9C), the distance of the closest astrocytic process extension to a dendritic spine will be measured (random sampling n=˜ 20) interactions/neuron; via 3D z-stacks); i.e., linear distance between the tip of the astrocyte process to the apex of the dendritic spine head. This distance is expected to increase with astrocytic Rac1 knockout, as suggested by in vivo and post-mortem studies (Bourne et al., 2008; Grutzendler et al., 2011; Nishida et al., 2007; Perez-Alvarez et al., 2014). Current imaging resolution may preclude a more detailed glial-neural interaction study at this time. However, if these first observations reveal significant differences across groups, the use of more specialized imaging equipment, e.g., stimulated emission depletion (STED) microscopy or electron microscopy, will be explored.

(III) To Study the Combined Effect of Rac1 Knockout in Both Motor Neurons and Astrocytes after SCI

Rationale: Emerging evidence support a mechanistic framework of the involvement of Rac1 activity in an astrocyte-neuronal relationship, which underlies motor neuronal dendritic spine dysgenesis in spasticity after SCI (FIG. 4A-D). To investigate the possibility of the contribution of an astrocyte-motor neuronal relationship in spasticity after SCI, the Cre-Lox Rac1 knockout platforms described above will be combined.

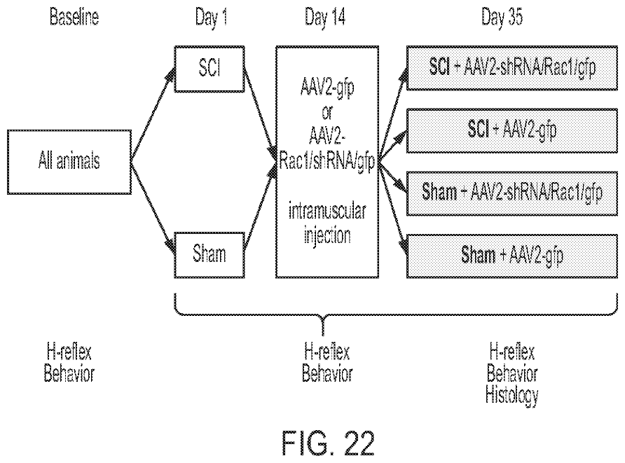

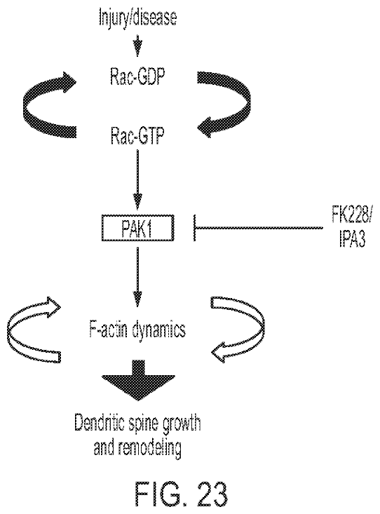

Study Design: Three animal groups will be prepared (male/female equal mix; 6-8 weeks old; n=30/group): 1) Sham (cre-lox driven tdTomato reporter)+AAV-Cre/gfp, 2) SCI (GFAP-Cre/Rac1^{f/f}/tdTomato)+AAV-Cre/gfp, 2) SCI (GFAP-Cre/tdTomato)+AAV-Cre/gfp. The same SCI contusion injury will be used to induce spasticity as detailed above. All animals have alleles for Cre-dependent tdTomato reporter expression. All transgenic animals are established breeding colonies in the laboratory (see FIG. 9A-D). For clarity, the genotypes for each group are shown in FIG. 22.

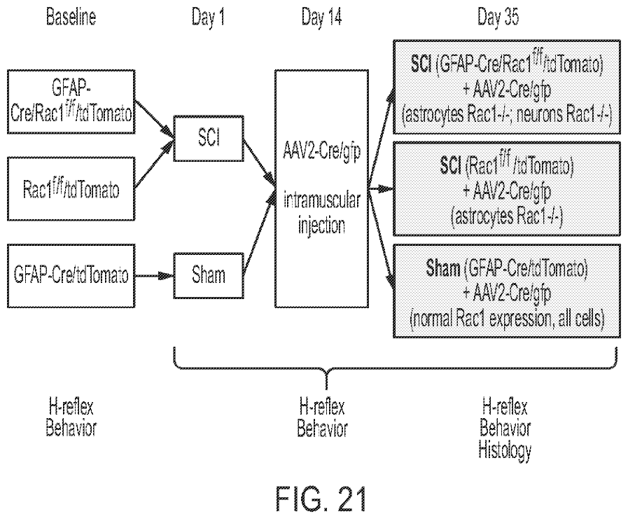

Two genotypes will be used for animals with SCI (FIG. 21): 1) GFAP-promoter driven Cre-lox Rac1 knockout (red astrocytes), or 2) Rac1 floxed background in which Cre is introduced through an intramuscular injection of AAV2-Cre/GFP that will knockout Rac1 in α-motor neurons (Rac1^{f/f}/tdTomato versus GFAP-Cre/Rac1^{f/f}/tdTomato). Because the Rac1^{f/f}/tdTomato allele is global in all animas, AAV2-Cre/GFP infected α-motor neurons will appear yellow in merged confocal images (i.e., co-localized tdTomato+GFP=yellow). Dendritic spines will be visible for analysis using GFP expression (FIG. 10C). A Sham, transgenic reporter mouse group that will express tdTomato reporter in astrocytes (normal Rac1 expression) and tdTomato/GFP in AAV-infected motor neurons, will be included. To ensure equivalent astrocyte labeling and histological analyses, all tissues will be immunostained for GFAP using an appropriate fluorescent color tag.

Outcome Measures: All outcome assessments are similarly as described above. To monitor other effects, including inflammatory response, brain and DRG tissues will be collected. To analyze dendritic spine morphology in reporter-labeled motor neurons, as well as other immuno-histological assessments, SWITCH cleared spinal cord samples will be used. To describe a putative interaction between astrocytes and neurons, the same approach as described above will be used. Outcome data will be compared across treatment groups using appropriate parametric or non-parametric statistical models, e.g. ANOVA or Rank-sum tests, and corrected for repeated measure errors post hoc, e.g., Bonferroni.

b. Research to Assess the Feasibility of Two Translationally-Relevant Approaches Targeting the Rac1-PAK1 Pathway to Relieve Spasticity

### (i) Investigate the Utility of a Gene Therapy “Platform” Targeting Rac1 to Reduce Spasticity

Rationale: In the United States, there are currently more than 54 active clinical trials (phase I to III) that use AAV gene therapy (US clinical trials website; accessed Oct. 20, 2017). Although none of these trials address SCI/D, viral-based therapeutic platforms have begun to emerge as a potential tool to address neurological disease, including within the spinal sensory or motor system, e.g. ALS (Azzouz, 2006). Two previous studies have shown that administration of an adeno-associated virus (AAV2 serotype) to deliver a knockdown construct can reduce sodium channel Nav1.3 misexpression and attenuate neuropathic pain after peripheral nerve injury or diabetic neuropathy (Samad et al., 2013; Tan et al., 2015a). Similar AAV-mediated tools have been shown to modify spinal motor neuron function (Boyce et al., 2012; Petruska et al., 2010; Towne et al., 2009). Importantly, studies have shown that these AAV2 vectors selectively infect neuronal tissue without damage to the CNS or chronic inflammation, e.g., low immunogenicity risk (Chamberlin et al., 1998; Finkelstein et al., 2001; Samad et al., 2013; Tan et al., 2015a). Here, the utility of a gene therapy to knockdown Rac1 will be assessed in α-motor neurons of animals with SCI-induced spasticity.

Study Design: Four animal groups will be prepared (male/female equal mix; 6-8 weeks old; n=30/group): 1) Sham+AAV2-GFP, 2) Sham+AAV2-shRNA/Rac1/GFP, 3) SCI+AAV2-GFP, and 4) SCI+AAV2-shRNA/Rac1/GFP. All mice are wild-type (C56/Blk6). AAV-shRNA constructs for Rac1 knockdown have been preliminarily validated (FIG. 10A-C). SCI and Sham groups will be prepared similarly as described above. The study design is shown in FIG. 22.

Outcome Measures: Testing will be performed at three time points: at baseline (before randomization and any surgeries), Day 14 post-SCI/Sham (before AAV injections), and Day 35. Electrophysiological and behavioral assessments for spasticity are the same as detailed above. A sub-population of animal spinal cord tissue will be used to assess the extent of Rac1 knockdown, e.g., Rac1-Pak1 pulldown assay, quantitative Western blot. To profile dendritic spine morphology on reporter-labeled motor neurons, as well as other immuno-histological assessments, e.g., inflammation, SWITCH clearing will be used on tissue samples. Confocal microscopy and the Neurolucida system will be used for image analysis of motor neurons and their dendritic spines (expressing GFP reporter protein). The spinal cord will be co-labeled with antibodies for motor neurons, e.g., ChaT, Rac1 (inactive/active total protein), iba-1 (microglia), GFAP (astrocytes), and other inflammatory markers. Changes in presynaptic terminal density will also be investigated in co-labeling studies with VGluT1 (Tan et al., 2012a). Outcome data will be compared across treatment groups using appropriate parametric or non-parametric statistical models, e.g. ANOVA or Rank-sum tests, and corrected for repeated measure errors, e.g., Bonferroni, Dunn's tests.

(II) Additional Studies to Assess the Potential of Targeting PAK1 with Romidepsin

Study Design: Four animal groups will be prepared (adult m/f mix; 6-8 weeks old; n=60/group): 1) Sham+DMSO/vehicle, 2) Sham+Romidepsin, 3) SCI+DMSO/vehicle, and 4) SCI+Romidepsin. All animals in this study will be reporter mice expressing fluorescent GFP driven by the neuron-specific thymus cell antigen-1 promotor (Thy1-GFP). Thy1-GFP mice are useful because of the reporter's specificity for neurons, clarity of dendritic spines in vivo, and are an available commercial stock from Jackson Laboratories. Romidepsin at MTD or DMSO vehicle will be administered as three intrathecal (i.t.) injections two-weeks after SCI or Sham surgeries. The experiment will be performed as a “vehicle-controlled crossover study” (see FIG. 24). This study design leverages the expected transient effect of romidepsin on spasticity due to the drug's relatively short half-life (<10) hours) based on published PK/PD studies (Berg et al., 2004). As such, this design space should reveal a close functional relationship between romidepsin treatment, and attenuated dendritic spine dysgenesis and spasticity.

The maximum tolerated dose (MTD) for romidepsin has been established as detailed above (once/day for 3 days: 10 μl per IP injection at 1.25 mg/kg; diluted in 1% DMSO; FIG. 12A-B and FIG. 13). The initial dosages in the pilot dose-response study were calculated using FDA guidelines for converting drug dosage between species (animal mg/kg dose× animal km=human mg/kg dose×human km) from the human MTD for lymphoma treatment (FDA website, last accessed on Nov. 21, 2017). Vehicle (1% DMSO) will be used as a control treatment.

Outcome Measures: Blinded observers will perform all physiological and behavioral assessments for spasticity and locomotor function, i.e., H-reflex, swim-testing, BMS, CatWalk, at six time points: at baseline, Day 11, Day 15, Day 25, Day 29, and Day 35. To ensure equivalency across testing, all physiological and behavioral testing is performed <24 hours immediately before the first, or after, the last drug treatment dose (i.e., Day 11/15 and Day 25/29; FIG. 24). After functional testing, a subpopulation of animals from each group will be removed for tissue collection at endpoint Days 14, 28, and 35 post-SCI/Sham surgery.

To monitor secondary effects of romidepsin treatment, body weight will be monitored on a weekly basis as an indicator for overall animal well-being. A major contribution to overall well-being can be extrapolated from human romidepsin studies, which show that the most common adverse events can directly impact body weight, e.g., loss of appetite, change in taste sensation, lack of strength, fatigue, and diarrhea (Celgene website, full prescribing information for Istodax, aka romidepsin, accessed Nov. 7, 2017). General animal activity will also be monitored in their familiar home environment using an “ActivMeter” (BioSeb), a device that automatically measures a cage's vibrations. ActivMeter tests will be run weekly over a 24-hour period (a single circadian cycle) on singly-housed animals with multiple comparator groups simultaneously (Charlet et al., 2011). Similar to humans in poor health, e.g., chronic pain, it is expected animals with drug complications will exhibit less activity within their “home” environment than compared with control animals.

### 4. Evaluation of Romidepsin for Treatment of Neuropathic Pain

Based on the literature, concentrations of romidepsin at 0.1-1 nM significantly reduces PAK1 kinase activity (Hirokawa et al., 2005; Maruta, 2011). Romidepsin and its active metabolites can passively penetrate through the blood brain barrier (BBB) in relatively low concentrations when administered systemically in non-human primates or rats (Berg et al., 2004). Depending on the organism, romidepsin may have a short half-life of <10 hrs (Berg et al., 2004). Potentially much higher CNS bioavailability can be possible after nervous system injury or intrathecal administration (Matsushita et al., 2015).

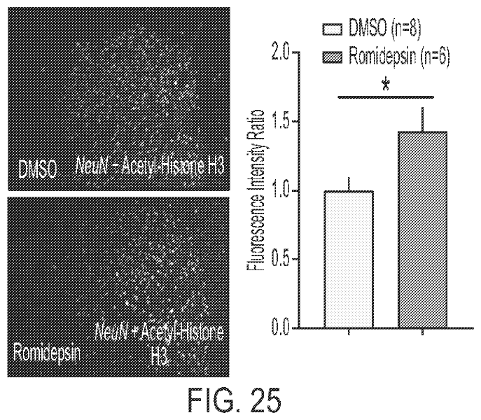

In a published burn study, whether romidepsin injected intraperitoneally could decrease burn-skin pain, e.g., an inflammatory pain model, and reverse dendritic spine dysgenesis (a presumed structural bioassay for pain), and reduced c-fos expression (e.g., a postmortem antigen marker for neuronal activity) was examined. To confirm tissue bioavailability, romidepsin was injected intraperitoneally, which seemed to penetrate through the BBB and resulted in an upregulation of histone acetylation in neurons within 24-hours post-administration (Guo et al., 2018; VanderMolen et al., 2011). Without wishing to be bound by theory, it is thought that the action of romidepsin as an analgesic is within the spinal cord nociceptive system, and when administered peripherally, does appear to have sufficient bioavailability within the spinal cord to produce a drug-tissue response (FIG. 25).

Based on published evidence and prior work, Pak1 inhibition with romidepsin is expected to prevent or reverse the presence of clinically intractable neuropathic pain through its disruption of abnormal dendritic spine remodeling within the spinal cord nociceptive/pain system.

Here, the use of “repurposed” romidepsin seeks to target PAK1, a downstream effector of Rac1 that links Rac1 to cytoskeletal reorganization and dendritic spine plasticity to effectively reduce/manage neuropathic pain. As proposed, romidepsin would act upon the “universal” PAK1 target for mitigating neuropathic pain that follows a spectrum of nervous system insults or diseases associated with neuropathy, i.e., nerve injury, spinal cord injury, multiple scelorsis (MS), diabetes, chemotherapy.

To first assess whether romidepsin has efficacy against neuropathic pain, the drug is administered in two established animal models of peripheral nerve injury: a spared-nerve injury (SNI) and chronic constriction injury (CCI) (Benson et al., 2020; Cichon et al., 2018; Gopalsamy et al., 2019; Karl et al., 2019).

a. Peripheral Nerve Injury Models (SNI and CCI)

It was determined to test romidepsin in the SNI and CCI nerve injury models to form a comprehensive dataset of the drug's effect on nerve injury induced-pain outcome. Although SNI and CCI produce neuropathic pain phenotype, they do so through differing mechanisms with distinct time courses for maximal pain onset.

An SNI is a transection-type peripheral nerve injury model, whereby a portion of the sciatic nerve innervating the hindlimb is ligated and cut. In this nerve injury model, the common peroneal and tibial nerves are injured, producing consistent and reproducible pain hypersensitivity in the cutaneous territory of the spared sural nerve. Additionally, the SNI leads to prolonged mechanical and thermal hyperresponsiveness in behavioral studies, which closely mimic clinically-described neuropathic pain conditions (Cichon et al., 2018). With an SNI, adult mice rapidly develop maximal neuropathic pain sensitivity within 3 days post-SNI (Benson et al., 2020; Cichon et al., 2018).

In a CCI is a chronic compression-type peripheral nerve injury model, whereby the sciatic nerve tract is compressed with ligatures, e.g., no cut/severing of nerve tissue. CCI in mice progressively leads to an inflammatory reaction and a subsequent severe loss of large, myelinated fibers, and ultimately peripheral chronic pain. With a CCI, neuropathic pain symptoms develops more slowly than in the SNI model, reaching maximal pain sensitivity around 14-days post-CCI (Gopalsamy et al., 2019; Karl et al., 2019; Tan et al., 2011).

Pak1-inhibition via romidepsin should act to attenuate both SNI and CCI induced pain: CCI and SNI peripheral nerve injury models in mice produce significant physiological signs and behavioral symptoms of clinical-like neuropathic pain, e.g., thermal hyperalgesia, punctate/pressure-induced mechanical allodynia. Additionally, it has been previously shown that both SNI and CCI lead to abnormal Rac1-regulated plasticity of dendritic spines in the spinal cord nociceptive neurons (Samad et al., 2013: Tan et al., 2011). As such, the proposed mechanism-of-action for romidepsin PAK1-inhibition should act similarly to attenuate pain in SNI and CCI.

b. Efficacy of Romidepsin in Nerve-Induced Chronic Pain

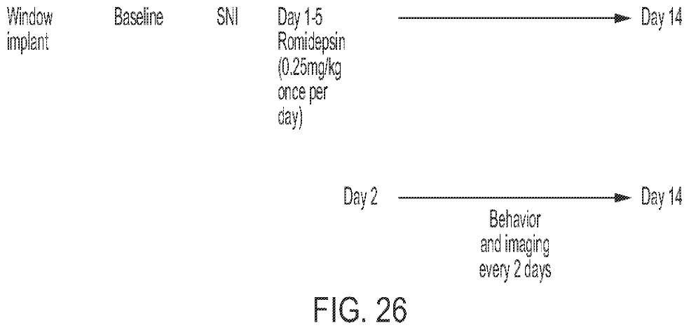

To evaluate the efficacy of romidepsin in treating nerve-induced chronic pain, the following comparator groups were prepared (weight-matched, adult male/female equally mixed C57BL/6 mice): Sham (n=5), SNI+Vehicle (n=5), and SNI+romidepsin (n=5). Romidepsin was administered as an intraperitoneal (IP) injection for five consecutive days after SNI (once/day for 5 days: 5 μl per injection at 0.25 mg/kg). Vehicle administration route was the same as romidepsin (1% diluted in DMSO; IP route), and sham animals underwent all procedures except for nerve injury. See FIG. 26.

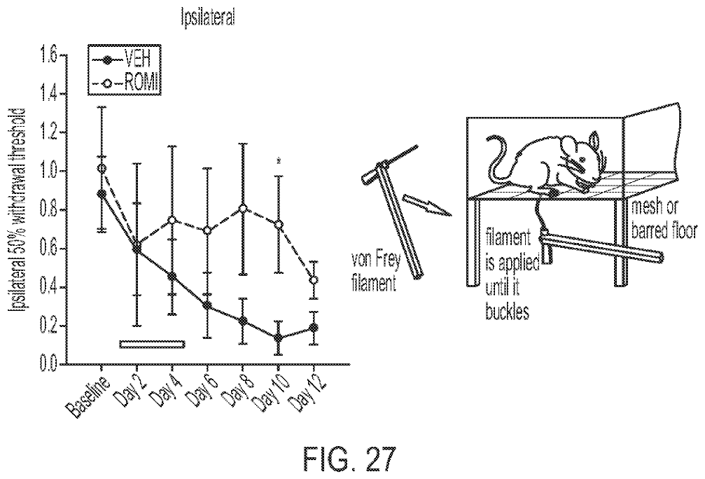

As illustrated in FIG. 27, administration of romidepsin immediately after a peripheral nerve injury (i.e., a mouse spared nerve injury aka SNI pain model) resulted in a significant decrease in mechanical-evoked pain.

c. Early Tolerability and Safety Testing

In adult mice, no observable adverse effects from romidepsin treatment were observed at the doses evaluated in ambulation, e.g., movement, body mass loss, or other outward signs of drug-induced toxicity/adverse events, as compared with untreated, control mice. Food intake appeared the same between romidepsin treated animals and controls with or without nerve injury.

d. Future Research to Evaluate Romidepsin Treatment in Peripheral Nerve Injury-Induced Neuropathic Pain

### (i) Drug Dosing and Safety

Without wishing to be bound by theory, the goal is to provide proof-of-concept that targeting PAK1 with romidepsin can prevent or reverse the presence of neuropathic pain. To determine the maximum tolerated dose (MTD) in the nerve injury models, i.e., SNI and CCI, the following comparator groups will be prepared (weight-matched, adult male/female equally mixed C57BL/6 mice): Sham (n=15), SNI or CCI+Vehicle (n=15/injury model), and SNI+romidepsin at three doses (1×, 2×, 3×; n=15 per dose concentration/per injury model). Romidepsin will be administered as three intrathecal (IT) injections two-weeks after SNI (once/day for 14 days: 5 μl per injection at 0.25 mg/kg (1×), 0.5 mg/kg (2×), and 0.75 mg/kg (3×)). Vehicle administration route will be the same as romidepsin (1% diluted in DMSO; IT route), and sham animals will undergo all procedures except for nerve injury.

The MTD of romidepsin administered via non-surgical IT injections, once daily for 14 days, that animals can tolerate without exhibiting adverse signs will be determined, including: a) weight loss within a one-week time frame of more than 10% of baseline, b) general home cage activity that progressively and significantly decreases over time, as compared with baseline uninjured, control activity measures, and c) failure to recover at any time point after romidepsin injection (measured by the Bioseb ActivMeter device shown below).

Rationale for Dosing: These initial romidepsin dosages were determined on two interrelated rationales. 1) The FDA guidelines for converting drug dosage between human and animal (animal mg/kg dose× animal km=human mg/kg dose×human km) from the maximum-tolerated dose (MTD) indicated for romidepsin for human cancer treatment (FDA website, accessed Apr. 11, 2021) were used. 2) Because it is expected that the utility of romidepsin for pain mitigation would likely require longer-term use than for the oncology indication, a pilot study in mice was performed with a lower dose of romidepsin for a prolonged period of time (once daily injections up to 14 days). At this lower initial dose (0.25 mg/kg), adverse side effects based on two blinded investigators' visual observation of general motor behavior (e.g., open field ambulation) and other visual measures of animal well-being, e.g., grooming, normal cage activities, feeding, etc, were not observed.

If romidepsin treatment shows lower efficacy in one nerve injury model compared with the other, the dosing strategy can be adjusted, e.g., concentration/volume, injection frequency, or focus our effort on a single nerve injury model at that time at that time. As appropriate, systemic romidepsin spread would continue to be monitored, and attempt to drug bioavailability restricted to local spinal cord tissues.

### (ii) Initial Monitoring for Adverse Effects

There are already a number of documented adverse events associated with romidepsin administered in humans at maximum tolerated dose (MTD) for its indicated use for oncologic diseases (BMS full prescribing information for Istodax, website accessed Apr. 8, 2021). In clinical trials, the most common adverse reactions (all grades) were neutropenia, lymphopenia, thrombocytopenia, infections, nausea, fatigue, vomiting, anorexia, anemia, and ECG T-wave changes. Adverse reactions (grade 3 or 4) that would most likely confound pain outcomes in the rodent study were rare (<2-8% occurrence) in two human trial studies of romidepsin, and include issues related weight loss, e.g., due to GI disorder, and fatigue.

For the proposed proof-of-concept study with romidepsin for neuropathic pain, a dosing strategy may involve longer term treatment, e.g., more doses at lower drug concentrations, compared to that with the oncology treatment regimen. Thus, animal body weight will be continually profiled over the courses of the study, and any adverse reactions noted, such as lethargic behavior, that could develop over time with romidepsin treatment.

It is also recognized that the inhibition of Pak1 may have unexpected effects on axonal plasticity or regeneration after nerve injury and may functionally affect regions of the CNS. In postmortem tissues, the potential effects of romidepsin on aberrant axonal plasticity will be assessed, with methods described previously, e.g., tract-tracing, electrical conduction studies (Tan et al., 2012a: Tan et al., 2006; Tan et al., 2007). To assess potential issues related to higher cortical level function due to romidepsin dosing in the animal study, cognitive behavioral tests will be performed in animal groups. Three assessments will be included for cognitive effect of romidepsin treatment, which will be tested weekly during the study (see below): 1) “2-object novel object recognition” evaluates cognition, particularly recognition memory, in rodents of CNS disorders. In this test, rodents spontaneously spend more time exploring novel objects than a familiar one. The choice to explore a novel object reflects the use of learning and memory (i.e., recognition). 2) “Morris water maze” tests long-term spatial memory wherein a rodent must remember where the hidden underwater platform is located based on visual spatial cues around the testing area. 3) “Rearing behavior” is associated with general activity level and has been used as a measure of higher order, cognitive-affective/exploratory function in animal models (i.e., more rearing events indicates higher levels of general activity and exploration) (Adams et al., 1985: Sheets et al., 2013).

Brain tissue will be collected to examine the effects of nerve injury and/or romidepsin in the cortex, hippocampus, and anterior cingulate cortex (ACC: a region associated with higher-level function, such as emotion). It is unclear how systemic romidepsin may affect these areas. Furthermore, exploratory assessments include histological studies for inflammatory response, and H3 histone acetylation for drug-tissue activity in different areas of the body, e.g., vital organs.

Future Consideration for Follow-Up Evaluation: If romidepsin treatment via the IT route demonstrates efficacy in attenuating pain in SNI and/or CCI nerve injury models without significant adverse effects or tolerability issues, the study's endpoint will be extended. To extend experimental endpoints and study drug treatment for several additional weeks beyond the initial nerve injury (e.g., +12 weeks), dosing will be adjusted by pursuing more advanced long-term treatment methods, such as using slow-release substrates or osmotic mini-pumps. Follow-up pharmacokinetic/pharmacodynamic (PD/PK) evaluation of romidepsin treatment will also be performed (Berg et al., 2004: Hirokawa et al., 2005).

(iii) Assessing Romidepsin Bioavailability

To monitor in vivo drug-response using established clinical biomarkers from all animals before and after intrathecal treatment with romidepsin at MTD, histone acetylation in peripheral blood mononuclear cells (PBMCs) or lymphocytes/monocytes in CSF (i.e., ELISA pharmacodynamics assessment) will be assessed (Cotto et al., 2010; VanderMolen et al., 2011). To monitor drug-tissue effect on spinal cord and brain tissue directly, histology will be performed to detect p-Raf and Acetyl-Histone H3 on sampled post-mortem tissue from these regions, as previously performed (Guo et al., 2018). Romidepsin is a histone deacetylase (HDAC) inhibitor, which leads to a potent block of Pak1 kinase activity. Raf-1 is a downstream effector of Pak1, and romidepsin activity in a tissue would result in a decrease of activated Raf-1, that is, phosphorylation of Ser338 on Raf-1, or p-Raf expression (Guo et al., 2018: Kalwat et al., 2013). Collectively, an increase in histone acetylation and decreased p-Raf in romidepsin-exposed CNS tissues following drug treatment is expected (Guo et al., 2018).

### (iv) Efficacy in Pain Outcome

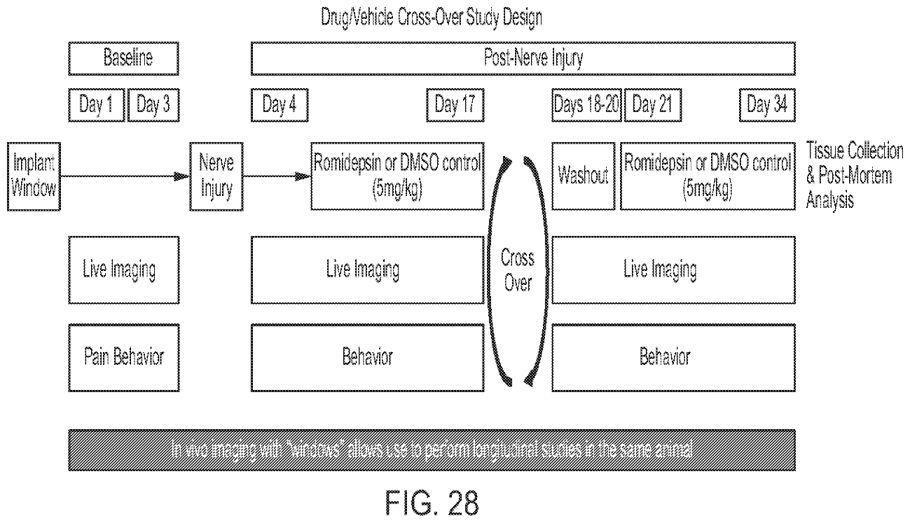

SNI and CCI nerve injured animal cohorts will be studied in a two distinct, independently run series of studies (i.e., not in-parallel) using the same study design. To test the efficacy of romidepsin, established preclinical assessments will be used in animal neuropathic pain models (SNI and CCI nerve injury). Here, a vehicle-controlled cross-over study design will be used (FIG. 28).

This longitudinal study design shown in FIG. 28 leverages the intravital imaging system, which allows for structural changes in the spinal cord nociceptive region over time in the same animal (before and after drug treatment) to be studied (Benson et al., 2020). Moreover, because a transient effect of romidepsin is expected on pain outcome due to the drug's relatively short half-life (<10 hours, as shown in published human PK/PD studies) (Berg et al., 2004), this study design should reveal a close functional relationship between romidepsin treatment, and attenuated pain outcome with the associated dendritic spine reorganization in the spinal cord nociceptive system.

To test the efficacy of romidepsin at MTD, a mouse expressing fluorescent reporter in neurons (Thy1-YFP: adult m/f) will be used in models of SNI and CCI (Samad et al., 2013). Studies will be performed on three comparator groups: Sham+romidepsin (not shown in FIG. 28; n=15), nerve-injury+vehicle (n=15/injury type), and nerve injury+romidepsin (n=15/injury type).

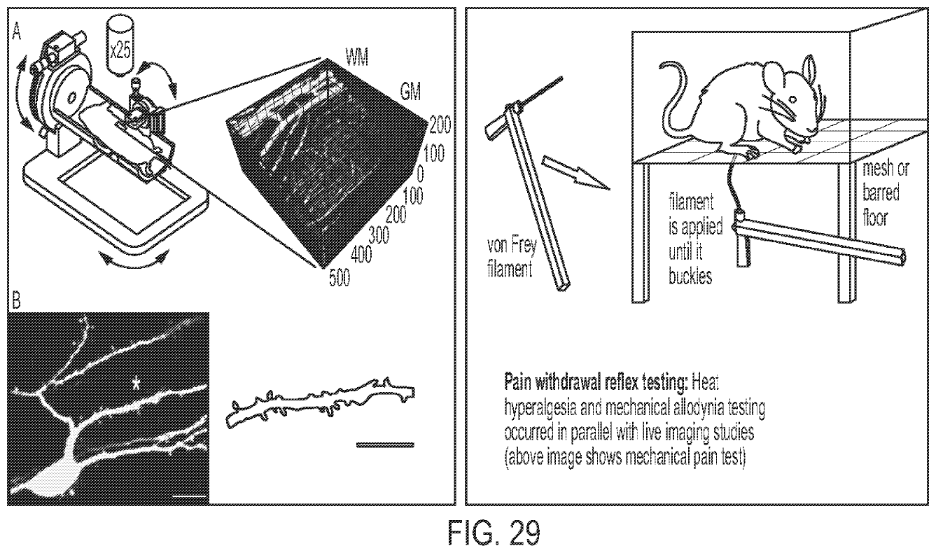

Published in vivo two-photon imaging protocol will be implemented to assess dynamic dendritic spine changes as a structural bioassay for neuropathic pain (Benson et al., 2020). In addition to in vivo imaging, functional testing for pain-related behavioral outcomes will be performed (FIG. 29—left/right panel). This includes pain sensitivity testing for heat hyperalgesia, i.e., Hargreaves, paw withdrawal testing, and mechanical/tactile allodynia, i.e., punctate graded Von Frey filament tests (Benson et al., 2020; Tan et al., 2012b).

It is recognized that there are limitations in animal pain-behavior testing methods. There may be concern that hind paw withdrawal response is not an accurate indicator of nociception, but represents an alteration in locomotion or hyperreflexia that accompany SCI. It will be ensured that evoked nocifensive behavior is accompanied by complex supraspinal behaviors, such as abrupt head turns, avoidance behaviors, and vocalizations, that are consistent with the interpretation that a previously innocuous stimulus has become noxious. To avoid an over-reliance on pain-reflex withdrawal testing, which do not always translate to the clinical setting, three additional assessments for pain severity will also be performed as follows:

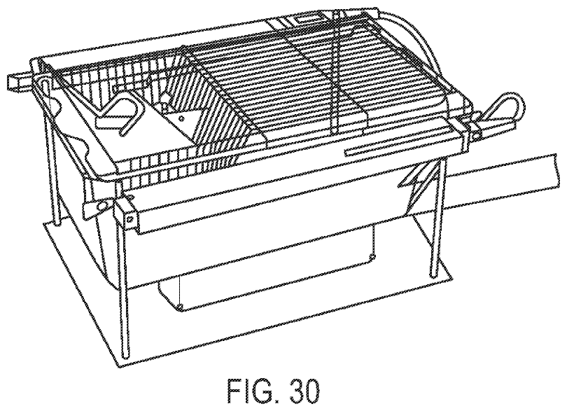

A) An “ActivMeter” (BioSeb) will be used. This is a device that automatically measures a cage's vibrations to accurately assess an animal's activity in its familiar environment (FIG. 30). Activity-recording tests will be run once per week over a 24 hour period (a single circadian cycle) on singly-housed animals with multiple comparator groups simultaneously (Charlet et al., 2011). Similar to humans in severe pain, it is expected that animals in excessive pain will exhibit less general activity within their “home” environment than compared with animals in less pain. It is acknowledged that animal activity may be affected by adverse events with romidepsin treatment. However, it is important to note that a significant loss of activity is not expected with drug dosing at MTD alone, which would be established in dosing studies (detailed above) prior to efficacy assessments. Nonetheless, this possibility will be contemplated during data interpretation of any significant decrease from control-treated, nerve-injured animal cohorts in 24-hour activity profiles.

2) To complement pain assessments, an Escape-Avoidance Task will be performed, which has been used to examine the affective-emotional component of pain (Pratt et al., 2013). In this task, animals are placed in a chamber separated into two compartments. In this pain-aggravated assessment, rodents learn to avoid an environment where an adverse stimulus, e.g., foot shock, is previously delivered. The latency between the stimulus application and the animal moving to escape/avoid the stimulus will be measured and compared across treatment groups (LaBuda et al., 2000; Vorhees et al., 2014). Without wishing to be bound by theory, it is expected that animals with lower pain threshold will have a lower escape latency.

3) Finally, terminal (non-survival) electrophysiology will be used to assess nociceptive hyperexcitability, e.g., the presence of central sensitization, with or without romidepsin administration. These electrophysiological studies will be conducted in ipsilateral dorsal horn nociceptive neurons in the spinal cord with whole animal preparations, as previously performed (Chang et al., 2010); Samad et al., 2013; Tan et al., 2011). In animals with ongoing neuropathic pain, stimulus-evoked hyperexcitability is expected in spinal nociceptive tissues, i.e., as a result of skin pinching and punctate von Frey testing of the innervating cutaneous receptive field. In contrast, it is expected that romidepsin treatment to attenuate pain will reduce excessive single unit firing as stimulus-evoked response.

At the end of all efficacy studies, tissue will be collected for histological analyses. Response to romidepsin will be evaluated in spinal cord and brain tissue (as described above), inflammation, e.g., microgliosis/astrogliosis, and morphological reorganization of neuronal dendritic spines in nociceptive neurons in spinal cord.

e. Summary of Animal Use and Statistics

A total of 210 mice will be required for this project. In the drug dosing and safety assessment component, it is estimated that 135 adult weight-matched wild-type C56/Blk6 mice (10-12 weeks old, male and female mix) will be needed. These animals will be assigned to comparator groups to confirm MTD using a dose-response study along with a series of behavioral tests. In the efficacy component, the need for 75 adult weight-matched Thy1-YFP transgenic mice (10)-12 weeks old, male and female mix) is estimated. These animals will undergo live imaging studies as well as pain-related behavioral testing in the Study Design shown in FIG. 28. Overall, the rationale for the proposed group sample sizes is based upon a power analysis of major outcomes (anatomy and behavior), and the expected needs from prior published studies. The a priori power analysis for this project is based on the use of G*Power 3.1.9.6 software for Mac OSX 11.2.3.

All statistical comparisons will be performed at the a-level of significance of 0.05 by two-tailed analyses using parametric or non-parametric tests, as appropriate. Note that data from male and female animals will be analyzed two ways. Datasets will be pooled from both sexes prior to data analysis (e.g., pool m/f datasets), and datasets from each sex dataset analyzed separately (within m/f), across treatment groups. Statistical modeling will be applied for comparative measures analysis of variance (ANOVA) and Kruskal-Wallis one-way ANOVA on ranks, and Bonferroni's or Dunn's post hoc analysis used, respectively, to correct for repeated measure errors. Romidepsin efficacy data will be compared against data from uninjured Sham, and nerve-injured, vehicle treated animals.

### G. REFERENCES

### 1. as to Treatment of Spasticity

Adams, M. M., Hicks, A. L. Spasticity after spinal cord injury. Spinal Cord 43: 577-586, 2005
Ahmed, Z. Trans-spinal direct current stimulation alters muscle tone in mice with and without spinal cord injury with spasticity. J Neurosci 34: 1701-1709, 2014
Ashby, P., Mailis, A., Hunter, J. The evaluation of “spasticity”. Can J Neurol Sci 14: 497-500, 1987
Azzouz, M. Gene Therapy for ALS: progress and prospects. Biochim Biophys Acta 1762: 1122-1127, 2006
Baker-Herman, T. L., Fuller, D. D., Bavis, R. W., Zabka, A. G., Golder, F. J., Doperalski, N. J., Johnson, R. A., Watters, J. J., Mitchell, G. S. BDNF is necessary and sufficient for spinal respiratory plasticity following intermittent hypoxia. Nat Neurosci 7: 48-55, 2004
Bandaru, S. P., Liu, S., Waxman, S. G., Tan, A. M. Dendritic spine dysgenesis contributes to hyperreflexia after spinal cord injury. J Neurophysiol 113: 1598-1615, 2015
Basso, D. M., Fisher, L. C., Anderson, A. J., Jakeman, L. B., McTigue, D. M., Popovich, P. G. Basso Mouse Scale for locomotion detects differences in recovery after spinal cord injury in five common mouse strains. J Neurotrauma 23: 635-659, 2006
Benson, C., Hill, M., Liu, S., Dib-Hajj, S. D., Waxman, S. G., Tan, A. M. Gene therapy to alleviate hyperreflexia after spinal cord injury. Society for Neuroscience—Abstract 676. 14 2017
Berg, S. L., Stone, J., Xiao, J. J., Chan, K. K., Nuchtern, J., Dauser, R., McGuffey, L., Thompson, P., Blaney, S. M. Plasma and cerebrospinal fluid pharmacokinetics of depsipeptide (FR901228) in nonhuman primates. Cancer Chemother Pharmacol 54: 85-88,
Bertino, E. M., Otterson, G. A. Romidepsin: a novel histone deacetylase inhibitor for cancer. Expert Opin Investig Drugs 20: 1151-1158, 2011
Boda, B., Jourdain, L., Muller, D. Distinct, but compensatory roles of PAK1 and PAK3 in spine morphogenesis. Hippocampus 18: 857-861, 2008
Boulenguez, P., Liabeuf, S., Bos, R., Bras, H., Jean-Xavier, C., Brocard, C., Stil, A., Darbon, P., Cattaert, D., Delpire, E., Marsala, M., Vinay, L. Down-regulation of the potassium-chloride cotransporter KCC2 contributes to spasticity after spinal cord injury. Nat Med 16: 302-307, 2010
Bourne, J. N., Harris, K. M. Balancing structure and function at hippocampal dendritic spines. Annu Rev Neurosci 31: 47-67, 2008
Boyce, V. S., Park, J., Gage, F. H., Mendell, L. M. Differential effects of brain-derived neurotrophic factor and neurotrophin-3 on hindlimb function in paraplegic rats. Eur J Neurosci 35: 221-232, 2012
Brockett, A. T., LaMarca, E. A., Gould, E. Physical exercise enhances cognitive flexibility as well as astrocytic and synaptic markers in the medial prefrontal cortex. PLOS One 10:e0124859, 2015
Brooks, P. J., Tagle, D. A., Groft, S. Expanding rare disease drug trials based on shared molecular etiology. Nat Biotechnol 32: 515-518, 2014
Brooks, P. J., Yang, N. N., Austin, C. P. Gene Therapy: The View from NCATS. Hum Gene Ther 27: 7-13, 2016
Calabrese, B., Halpain, S. Differential targeting of dynamin-1 and dynamin-3 to nerve terminals during chronic suppression of neuronal activity. Mol Cell Neurosci 68: 36-45, 2015
Calabrese, B., Wilson, M. S., Halpain, S. Development and regulation of dendritic spine synapses. Physiology (Bethesda) 21: 38-47, 2006
Carp, J. S., Tennissen, A. M., Chen, X. Y., Wolpaw, J. R. H-reflex operant conditioning in mice. J Neurophysiol 96: 1718-1727, 2006
Chakrabarty, S., Friel, K. M., Martin, J. H. Activity-dependent plasticity improves M1 motor representation and corticospinal tract connectivity. J Neurophysiol 101: 1283-1293, 2009
Chamberlin, N. L., Du, B., de Lacalle, S., Saper, C. B. Recombinant adeno-associated virus vector: use for transgene expression and anterograde tract tracing in the CNS. Brain Res 793: 169-175, 1998
Chevy, Q., Heubl, M., Goutierre, M., Backer, S., Moutkine, I., Eugene, E., Bloch-Gallego, E., Levi, S., Poncer, J. C. KCC2 Gates Activity-Driven AMPA Receptor Traffic through Cofilin Phosphorylation. J Neurosci 35: 15772-15786, 2015
Corbetta, S., Gualdoni, S., Ciceri, G., Monari, M., Zuccaro, E., Tybulewicz, V. L., de Curtis, I. Essential role of Rac1 and Rac3 GTPases in neuronal development. FASEB J 23: 1347-1357, 2009
Cotto, M., Cabanillas, F., Tirado, M., Garcia, M. V., Pacheco, E. Epigenetic therapy of lymphoma using histone deacetylase inhibitors. Clin Transl Oncol 12: 401-409, 2010
Eaton, M. Common animal models for spasticity and pain. J Rehabil Res Dev 40: 41-54, 2003
Finkelstein, R., Baughman, R. W., Steele, F. R. Harvesting the neural gene therapy fruit. Mol Ther 3: 3-7, 2001
Fouad, K., Bennett, D. J., Vavrek, R., Blesch, A. Long-term viral brain-derived neurotrophic factor delivery promotes spasticity in rats with a cervical spinal cord hemisection. Front Neurol 4: 187, 2013
Glebov, O. O., Tigaret, C. M., Mellor, J. R., Henley, J. M. Clathrin-independent trafficking of AMPA receptors. J Neurosci 35: 4830-4836, 2015
Grutzendler, J., Yang, G., Pan, F., Parkhurst, C. N., Gan, W. B. Transcranial two-photon imaging of the living mouse brain. Cold Spring Harb Protoc 20112011
Guo, Y., Benson, C., Hill, M., Henry, S., Effraim, P., Waxman, S. G., Dib-Hajj, S., Tan, A. M. Therapeutic potential of Pak1 inhibition for pain associated with cutaneous burn injury. Mol Pain 2018
Halassa, M. M., Fellin, T., Haydon, P. G. The tripartite synapse: roles for gliotransmission in health and disease. Trends Mol Med 13: 54-63, 2007
Halpain, S., Spencer, K., Graber, S. Dynamics and pathology of dendritic spines. Prog Brain Res 29-37, 2005
Harward, S. C., Hedrick, N. G., Hall, C. E., Parra-Bueno, P., Milner, T. A., Pan, E., Laviv, T., Hempstead, B. L., Yasuda, R., McNamara, J. O. Autocrine BDNF-TrkB signalling within a single dendritic spine. Nature 538: 99-103, 2016
Hashizume, K., Kanda, K., Burke, R. E. Medial gastrocnemius motor nucleus in the rat: age-related changes in the number and size of motoneurons. J Comp Neurol 269: 425-430, 1988
Hayashi, K., Ohshima, T., Hashimoto, M., Mikoshiba, K. Pak1 regulates dendritic branching and spine formation. Dev Neurobiol 67: 655-669, 2007
Heasman, S. J., Ridley, A. J. Mammalian Rho GTPases: new insights into their functions from in vivo studies. Nat Rev Mol Cell Biol 9: 690-701, 2008
Hefferan, M. P., Kucharova, K., Kinjo, K., Kakinohana, O., Sekerkova, G., Nakamura, S., Fuchigami, T., Tomori, Z., Yaksh, T. L., Kurtz, N., Marsala, M. Spinal astrocyte glutamate receptor 1 overexpression after ischemic insult facilitates behavioral signs of spasticity and rigidity. J Neurosci 27: 11179-11191, 2007
Hirokawa, Y., Nakajima, H., Hanemann, C. O., Kurtz, A., Frahm, S., Mautner, V., Maruta, H. Signal therapy of NF1-deficient tumor xenograft in mice by the anti-PAK1 drug FK228. Cancer Biol Ther 4: 379-381, 2005
Holtz, K. A., Lipson, R., Noonan, V. K., Kwon, B. K., Mills, P. B. Prevalence and Effect of Problematic Spasticity After Traumatic Spinal Cord Injury. Arch Phys Med Rehabil 98: 1132-1138, 2017
Hoover, W. C., Zhang, W., Xue, Z., Gao, H., Chernoff, J., Clapp, D. W., Gunst, S. J., Tepper, R. S. Inhibition of p21 activated kinase (PAK) reduces airway responsiveness in vivo and in vitro in murine and human airways. PLOS One 7:e42601, 2012
Hultborn, H., Nielsen, J. B. Spinal control of locomotion—from cat to man. Acta Physiol (Oxf) 189: 111-121, 2007
Hunanyan, A. S., Petrosyan, H. A., Alessi, V., Arvanian, V. L. Combination of chondroitinase ABC and AAV-NT3 promotes neural plasticity at descending spinal pathways after thoracic contusion in rats. J Neurophysiol 110: 1782-1792, 2013
Impey, S., Davare, M., Lesiak, A., Fortin, D., Ando, H., Varlamova, O., Obrietan, K., Soderling, T. R., Goodman, R. H., Wayman, G. A. An activity-induced microRNA controls dendritic spine formation by regulating Rac1-PAK signaling. Mol Cell Neurosci 43: 146-156, 2010
Ishii, T., Ueyama, T., Shigyo, M., Kohta, M., Kondoh, T., Kuboyama, T., Uebi, T., Hamada, T., Gutmann, D. H., Aiba, A., Kohmura, E., Tohda, C., Saito, N. A Novel Rac1-GSPT1 Signaling Pathway Controls Astrogliosis Following Central Nervous System Injury. J Biol Chem 292: 1240-1250, 2017
Ji, R. R., Strichartz, G. Cell signaling and the genesis of neuropathic pain. Sci STKE 2004: reE14, 2004
Kalwat, M. A., Yoder, S. M., Wang, Z., Thurmond, D. C. A p21-activated kinase (PAK1) signaling cascade coordinately regulates F-actin remodeling and insulin granule exocytosis in pancreatic beta cells. Biochem Pharmacol 85: 808-816, 2013
Karakoyun, A., Boyraz, I., Gunduz, R., Karamercan, A., Ozgirgin, N. Electrophysiological and clinical evaluation of the effects of transcutaneous electrical nerve stimulation on the spasticity in the hemiplegic stroke patients. J Phys Ther Sci 27: 3407-3411, 2015
Kellner, Y., Godecke, N., Dierkes, T., Thieme, N., Zagrebelsky, M., Korte, M. The BDNF effects on dendritic spines of mature hippocampal neurons depend on neuronal activity. Front Synaptic Neurosci 6: 5, 2014
Kheder, A., Nair, K. P. Spasticity: pathophysiology, evaluation and management. Pract Neurol 289-298, 2012
Kichina, J. V., Goc, A., Al-Husein, B., Somanath, P. R., Kandel, E. S. PAK1 as a therapeutic target. Expert Opin Ther Targets 14: 703-725, 2010
Kim, B. G., Dai, H. N., McAtee, M., Vicini, S., Bregman, B. S. Remodeling of synaptic structures in the motor cortex following spinal cord injury. Exp Neurol 198: 401-415, 2006
Kitzman, P. VGLUT1 and GLYT2 labeling of sacrocaudal motoneurons in the spinal cord injured spastic rat. Exp Neurol 204: 195-204, 2007
Klein, R. L., Dayton, R. D., Tatom, J. B., Henderson, K. M., Henning, P. P. AAV8, 9, Rh10, Rh43 vector gene transfer in the rat brain: effects of serotype, promoter and purification method. Mol Ther 16: 89-96, 2008
Lance, J. W. The control of muscle tone, reflexes, and movement: Robert Wartenberg Lecture. Neurology 30: 1303-1313, 1980
Lee, H. J., Jakovcevski, I., Radonjic, N., Hoelters, L., Schachner, M., Irintchev, A. Better functional outcome of compression spinal cord injury in mice is associated with enhanced H-reflex responses. Exp Neurol 2009
Li, Y., Gorassini, M. A., Bennett, D. J. Role of persistent sodium and calcium currents in motoneuron firing and spasticity in chronic spinal rats. J Neurophysiol 91: 767-783, 2004a
Li, Y., Harvey, P. J., Li, X., Bennett, D. J. Spastic long-lasting reflexes of the chronic spinal rat studied in vitro. J Neurophysiol 91: 2236-2246, 2004b
Little, J. W., Ditunno, J. F., Jr. Soleus H-reflex in spastic spinal cord injury patients. Arch Phys Med Rehabil 85: 2070; author reply 2070-2071, 2004
Liu, F., Li, X., Wang, C., Cai, X., Du, Z., Xu, H., Li, F. Downregulation of p21-activated kinase-1 inhibits the growth of gastric cancer cells involving cyclin B1. Int J Cancer 125: 2511-2519, 2009
Llano, O., Smirnov, S., Soni, S., Golubtsov, A., Guillemin, I., Hotulainen, P., Medina, I., Nothwang, H. G., Rivera, C., Ludwig, A. KCC2 regulates actin dynamics in dendritic spines via interaction with beta-PIX. J Cell Biol 209: 671-686, 2015
Ma, Q. L., Yang, F., Frautschy, S. A., Cole, G. M. PAK in Alzheimer disease, Huntington disease and X-linked mental retardation. Cell Logist 2: 117-125, 2012
Maruta, H. Effective neurofibromatosis therapeutics blocking the oncogenic kinase PAK1. Drug Discov Ther 5: 266-278, 2011
Matsushita, T., Lankford, K. L., Arroyo, E. J., Sasaki, M., Neyazi, M., Radtke, C., Kocsis, J. D. Diffuse and persistent blood-spinal cord barrier disruption after contusive spinal cord injury rapidly recovers following intravenous infusion of bone marrow mesenchymal stem cells. Exp Neurol 267: 152-164, 2015
Murray, E., Cho, J. H., Goodwin, D., Ku, T., Swaney, J., Wedeen, V. J., Seung, H. S., Chung, K. Simple, Scalable Proteomic Imaging for High-Dimensional Profiling of Intact Systems. Cell 163: 1500-1514, 2015
Nakayama, A. Y., Harms, M. B., Luo, L. Small GTPases Rac and Rho in the maintenance of dendritic spines and branches in hippocampal pyramidal neurons. J Neurosci 20: 5329-5338, 2000
Nielsen, J. B., Crone, C., Hultborn, H. The spinal pathophysiology of spasticity—from a basic science point of view. Acta Physiol (Oxf) 189: 171-180, 2007
Nikolic, M. The Pak1 kinase: an important regulator of neuronal morphology and function in the developing forebrain. Mol Neurobiol 37: 187-202, 2008
Nishida, H., Okabe, S. Direct astrocytic contacts regulate local maturation of dendritic spines. J Neurosci 27: 331-340, 2007
Perez-Alvarez, A., Navarrete, M., Covelo, A., Martin, E. D., Araque, A. Structural and functional plasticity of astrocyte processes and dendritic spine interactions. J Neurosci 34: 12738-12744, 2014
Petruska, J. C., Kitay, B., Boyce, V. S., Kaspar, B. K., Pearse, D. D., Gage, F. H., Mendell, L. M. Intramuscular AAV delivery of NT-3 alters synaptic transmission to motoneurons in adult rats. Eur J Neurosci 2010
Racchetti, G., D'Alessandro, R., Meldolesi, J. Astrocyte stellation, a process dependent on Rac1 is sustained by the regulated exocytosis of enlargeosomes. Glia 60: 465-475, 2012
Raisman, G. Plasticity of adult central fibre tracts. Prog Brain Res 100: 195-201, 1994 Ryu, Y., Ogata, T., Nagao, M., Kitamura, T., Morioka, K., Ichihara, Y., Doi, T.,
Sawada, Y., Akai, M., Nishimura, R., Fujita, N. The swimming test is effective for evaluating spasticity after contusive spinal cord injury. PLOS One 12:e0171937, 2017
Samad, O. A., Tan, A. M., Cheng, X., Foster, E., Dib-Hajj, S. D., Waxman, S. G. Virus-mediated shRNA knockdown of Na (v) 1.3 in rat dorsal root ganglion attenuates nerve injury-induced neuropathic pain. Mol Ther 21: 49-56, 2013
Scholz, J., Woolf, C. J. The neuropathic pain triad: neurons, immune cells and glia. Nat Neurosci 10: 2007
Senger, D. L., Tudan, C., Guiot, M. C., Mazzoni, I. E., Molenkamp, G., LeBlanc, R., Antel, J., Olivier, A., Snipes, G. J., Kaplan, D. R. Suppression of Rac activity induces apoptosis of human glioma cells but not normal human astrocytes. Cancer Res 62: 2131-2140, 2002
Skold, C., Levi, R., Seiger, A. Spasticity after traumatic spinal cord injury: nature, severity, and location. Arch Phys Med Rehabil 80: 1548-1557, 1999
Stamboulian, S., Choi, J. S., Ahn, H. S., Chang, Y. W., Tyrrell, L., Black, J. A., Waxman, S. G., Dib-Hajj, S. D. ERK1/2 mitogen-activated protein kinase phosphorylates sodium channel Na (v) 1.7 and alters its gating properties. J Neurosci 30: 1637-1647, 2010
Tan, A. M. Dendritic spine dysgenesis in neuropathic pain. Prog Mol Biol Transl Sci 131: 385-408, 2015a
Tan, A. M. Dendritic spine dysgenesis: An emerging concept in neuropsychiatric disease. Neurosci Lett 601: 1-3, 2015b
Tan, A. M., Chakrabarty, S., Kimura, H., Martin, J. H. Selective corticospinal tract injury in the rat induces primary afferent fiber sprouting in the spinal cord and hyperreflexia. J Neurosci 32: 12896-12908, 2012a
Tan, A. M., Chang, Y. W., Zhao, P., Hains, B. C., Waxman, S. G. Rac1-regulated dendritic spine remodeling contributes to neuropathic pain after peripheral nerve injury. Exp Neurol 232: 222-233, 2011
Tan, A. M., Choi, J. S., Waxman, S. G., Hains, B. C. Dendritic spine remodeling after spinal cord injury alters neuronal signal processing. J Neurophysiol 102: 2396-2409,
Tan, A. M., Colletti, M., Rorai, A. T., Skene, J. H., Levine, J. M. Antibodies against the NG2 proteoglycan promote the regeneration of sensory axons within the dorsal columns of the spinal cord. J Neurosci 2006
Tan, A. M., Petruska, J. C., Mendell, L. M., Levine, J. M. Sensory afferents regenerated into dorsal columns after spinal cord injury remain in a chronic pathophysiological state. Exp Neurol 206: 257-268, 2007
Tan, A. M., Samad, O. A., Dib-Hajj, S. D., Waxman, S. G. Virus-Mediated Knockdown of Nav1.3 in Dorsal Root Ganglia of STZ-Induced Diabetic Rats Alleviates Tactile Allodynia. Mol Med 21: 544-552, 2015a
Tan, A. M., Samad, O. A., Liu, S., Bandaru, S., Zhao, P., Waxman, S. G. Burn injury-induced mechanical allodynia is maintained by Rac1-regulated dendritic spine dysgenesis. Exp Neurol 248: 509-519, 2013
Tan, A. M., Stamboulian, S., Chang, Y. W., Zhao, P., Hains, A. B., Waxman, S. G., Hains, B. C. Neuropathic pain memory is maintained by Rac1-regulated dendritic spine remodeling after spinal cord injury. J Neurosci 28: 13173-13183, 2008
Tan, A. M., Waxman, S. G. Spinal cord injury, dendritic spine remodeling, and spinal memory mechanisms. Exp Neurol 235: 142-151, 2012b
Tan, A. M., Waxman, S. G. Dendritic spine dysgenesis in neuropathic pain. Neurosci Lett 601: 54-60, 2015b
Tashiro, A., Yuste, R. Regulation of dendritic spine motility and stability by Rac1 and Rho kinase: evidence for two forms of spine motility. Mol Cell Neurosci 26: 429-440, 2004
Tenorio, G., Kulkarni, A., Kerr, B. J. Resident glial cell activation in response to perispinal inflammation leads to acute changes in nociceptive sensitivity. Pain 154: 71-81, 2013
Towne, C., Pertin, M., Beggah, A. T., Aebischer, P., Decosterd, I. Recombinant adeno-associated virus serotype 6 (rAAV2/6)-mediated gene transfer to nociceptive neurons through different routes of delivery. Mol Pain 5: 52, 2009
Towne, C., Raoul, C., Schneider, B. L., Aebischer, P. Systemic AAV6 delivery mediating RNA interference against SOD1: neuromuscular transduction does not alter disease progression in fALS mice. Mol Ther 16: 1018-1025, 2008
Valero-Cabre, A., Fores, J., Navarro, X. Reorganization of reflex responses mediated by different afferent sensory fibers after spinal cord transection. J Neurophysiol 91: 2838-2848, 2004
VanderMolen, K. M., McCulloch, W., Pearce, C. J., Oberlies, N. H. Romidepsin (Istodax, NSC 630176, FR901228, FK228, depsipeptide): a natural product recently approved for cutaneous T-cell lymphoma. J Antibiot (Tokyo) 64: 525-531, 2011
Walter, J. S., Sacks, J., Othman, R., Rankin, A. Z., Nemchausky, B., Chintam, R., Wheeler, J. S. A database of self-reported secondary medical problems among VA spinal cord injury patients: its role in clinical care and management. J Rehabil Res Dev 39: 53-61, 2002
Wang, Y., Lu, Y. F., Li, C. L., Sun, W., Li, Z., Wang, R. R., He, T., Yang, F., Yang, Y., Wang, X. L., Guan, S. M., Chen, J. Involvement of Rac1 signalling pathway in the development and maintenance of acute inflammatory pain induced by bee venom injection. Br J Pharmacol 173: 937-950, 2016
Watakabe, A., Ohtsuka, M., Kinoshita, M., Takaji, M., Isa, K., Mizukami, H., Ozawa, K., Isa, T., Yamamori, T. Comparative analyses of adeno-associated viral vector serotypes 1, 2, 5, 8 and 9 in marmoset, mouse and macaque cerebral cortex. Neurosci Res 93: 144-157, 2015
Wolpaw, J. R. Acquisition and maintenance of the simplest motor skill: investigation of CNS mechanisms. Med Sci Sports Exerc 26: 1475-1479, 1994
Yang, H., Mattingly, R. R. The Ras-GRFI exchange factor coordinates activation of H-Ras and Rac1 to control neuronal morphology. Mol Biol Cell 17: 2177-2189, 2006
Zhao, P., Hill, M., Liu, S., Chen, L., Bangalore, L., Waxman, S. G., Tan, A. M. Dendritic spine remodeling following early and late Rac1 inhibition after spinal cord injury: evidence for a pain biomarker. J Neurophysiol 115: 2893-2910, 2016

### 2. As to Treatment of Neuropathic Pain

Adams, L. M., Geyer, M. A. A proposed animal model for hallucinogens based on LSD's effects on patterns of exploration in rats. Behav Neurosci 99: 881-900, 1985
Asrar, S., Meng, Y., Zhou, Z., Todorovski, Z., Huang, W. W., Jia, Z. Regulation of hippocampal long-term potentiation by p21-activated protein kinase 1 (PAK1). Neuropharmacology 56: 73-80, 2009
Baker-Herman, T. L., Fuller, D. D., Bavis, R. W., Zabka, A. G., Golder, F. J., Doperalski, N. J., Johnson, R. A., Watters, J. J., Mitchell, G. S. BDNF is necessary and sufficient for spinal respiratory plasticity following intermittent hypoxia. Nat Neurosci 7: 48-55, 2004
Benson, C. A., Fenrich, K. K., Olson, K. L., Patwa, S., Bangalore, L., Waxman, S. G., Tan, A. M. Dendritic Spine Dynamics after Peripheral Nerve Injury: An Intravital Structural Study. J Neurosci 40: 4297-4308, 2020
Berg, S. L., Stone, J., Xiao, J. J., Chan, K. K., Nuchtern, J., Dauser, R., McGuffey, L., Thompson, P., Blaney, S. M. Plasma and cerebrospinal fluid pharmacokinetics of depsipeptide (FR901228) in nonhuman primates. Cancer Chemother Pharmacol 54: 85-88, 2004
Bertino, E. M., Otterson, G. A. Romidepsin: a novel histone deacetylase inhibitor for cancer. Expert Opin Investig Drugs 20: 1151-1158, 2011
Boda, B., Jourdain, L., Muller, D. Distinct, but compensatory roles of PAK1 and PAK3 in spine morphogenesis. Hippocampus 18: 857-861, 2008
Cao, X. C., Pappalardo, L. W., Waxman, S. G., Tan, A. M. Dendritic spine dysgenesis in superficial dorsal horn sensory neurons after spinal cord injury. Mol Pain 13: 1744806916688016, 2017
Chang, Y. W., Tan, A., Saab, C., Waxman, S. Unilateral focal burn injury is followed by long-lasting bilateral allodynia and neuronal hyperexcitability in spinal cord dorsal horn. J Pain 11: 119-130, 2010
Charlet, A., Rodeau, J. L., Poisbeau, P. Radiotelemetric and symptomatic evaluation of pain in the rat after laparotomy: long-term benefits of perioperative ropivacaine care. J Pain 12: 246-256, 2011
Chen, L. Y., Rex, C. S., Babayan, A. H., Kramar, E. A., Lynch, G., Gall, C. M., Lauterborn, J. C. Physiological activation of synaptic Rac>PAK (p-21 activated kinase) signaling is defective in a mouse model of fragile X syndrome. J Neurosci 30: 10977-10984, 2010
Cichon, J., Sun, L., Yang, G. Spared Nerve Injury Model of Neuropathic Pain in Mice. Bio Protoc 82018
Cotto, M., Cabanillas, F., Tirado, M., Garcia, M. V., Pacheco, E. Epigenetic therapy of lymphoma using histone deacetylase inhibitors. Clin Transl Oncol 12: 401-409, 2010
Gao, Y., Dickerson, J. B., Guo, F., Zheng, J., Zheng, Y. Rational design and characterization of a Rac GTPase-specific small molecule inhibitor. Proc Natl Acad Sci USA 101: 7618-7623, 2004
Gopalsamy, B., Sambasevam, Y., Zulazmi, N. A., Chia, J. S. M., Omar Farouk, A. A., Sulaiman, M. R., Tengku Mohamad, T. A. S., Perimal, E. K. Experimental Characterization of the Chronic Constriction Injury-Induced Neuropathic Pain Model in Mice. Neurochem Res 44: 2123-2138, 2019
Guo, Y., Benson, C., Hill, M., Henry, S., Effraim, P., Waxman, S. G., Dib-Hajj, S., Tan, A. M. Therapeutic potential of Pak1 inhibition for pain associated with cutaneous burn injury. Mol Pain 14: 1744806918788648, 2018
Hayashi, K., Ohshima, T., Hashimoto, M., Mikoshiba, K. Pak1 regulates dendritic branching and spine formation. Dev Neurobiol 67: 655-669, 2007
Hirokawa, Y., Arnold, M., Nakajima, H., Zalcberg, J., Maruta, H. Signal therapy of breast cancers by the HDAC inhibitor FK228 that blocks the activation of PAK1 and abrogates the tamoxifen-resistance. Cancer Biol Ther 4:956-960, 2005
Kalwat, M. A., Yoder, S. M., Wang, Z., Thurmond, D. C. A p21-activated kinase (PAK1) signaling cascade coordinately regulates F-actin remodeling and insulin granule exocytosis in pancreatic beta cells. Biochem Pharmacol 85: 808-816, 2013
Karl, F., Colaco, M. B. N., Schulte, A., Sommer, C., Uceyler, N. Affective and cognitive behavior is not altered by chronic constriction injury in B7-H1 deficient and wildtype mice. BMC Neurosci 20: 16, 2019
Kichina, J. V., Goc, A., Al-Husein, B., Somanath, P. R., Kandel, E. S. PAK1 as a therapeutic target. Expert Opin Ther Targets 14: 703-725, 2010
LaBuda, C. J., Fuchs, P. N. A behavioral test paradigm to measure the aversive quality of inflammatory and neuropathic pain in rats. Exp Neurol 163: 490-494, 2000
Liu, F., Li, X., Wang, C., Cai, X., Du, Z., Xu, H., Li, F. Downregulation of p21-activated kinase-1 inhibits the growth of gastric cancer cells involving cyclin B1. Int J Cancer 125: 2511-2519, 2009
Ma, Q. L., Yang, F., Frautschy, S. A., Cole, G. M. PAK in Alzheimer disease, Huntington disease and X-linked mental retardation. Cell Logist 2: 117-125, 2012
Maruta, H. Effective neurofibromatosis therapeutics blocking the oncogenic kinase PAK1. Drug Discov Ther 5: 266-278, 2011
Matsushita, T., Lankford, K. L., Arroyo, E. J., Sasaki, M., Neyazi, M., Radtke, C., Kocsis, J. D. Diffuse and persistent blood-spinal cord barrier disruption after contusive spinal cord injury rapidly recovers following intravenous infusion of bone marrow mesenchymal stem cells. Exp Neurol 267: 152-164, 2015
Pratt, D., Fuchs, P. N., Sluka, K. A. Assessment of avoidance behaviors in mouse models of muscle pain. Neuroscience 248: 54-60, 2013
Price, T. J., Rashid, M. H., Millecamps, M., Sanoja, R., Entrena, J. M., Cervero, F. Decreased nociceptive sensitization in mice lacking the fragile X mental retardation protein: role of mGluR 1/5 and mTOR. J Neurosci 27: 13958-13967, 2007
Samad, O. A., Tan, A. M., Cheng, X., Foster, E., Dib-Hajj, S. D., Waxman, S. G. Virus-mediated shRNA knockdown of Na (v) 1.3 in rat dorsal root ganglion attenuates nerve injury-induced neuropathic pain. Mol Ther 21: 49-56, 2013
Sheets, A. L., Lai, P. L., Fisher, L. C., Basso, D. M. Quantitative evaluation of 3D mouse behaviors and motor function in the open-field after spinal cord injury using markerless motion tracking. PLOS One 8:e74536, 2013
Tan, A. M. Dendritic spine dysgenesis in neuropathic pain. Prog Mol Biol Transl Sci 131:385-408, 2015
Tan, A. M., Chakrabarty, S., Kimura, H., Martin, J. H. Selective corticospinal tract injury in the rat induces primary afferent fiber sprouting in the spinal cord and hyperreflexia. J Neurosci 32: 12896-12908, 2012a
Tan, A. M., Chang, Y. W., Zhao, P., Hains, B. C., Waxman, S. G. Rac1-regulated dendritic spine remodeling contributes to neuropathic pain after peripheral nerve injury. Exp Neurol 232: 222-233, 2011
Tan, A. M., Colletti, M., Rorai, A. T., Skene, J. H., Levine, J. M. Antibodies against the NG2 proteoglycan promote the regeneration of sensory axons within the dorsal columns of the spinal cord. J Neurosci 26: 4729-4739, 2006
Tan, A. M., Petruska, J. C., Mendell, L. M., Levine, J. M. Sensory afferents regenerated into dorsal columns after spinal cord injury remain in a chronic pathophysiological state. Exp Neurol 206: 257-268, 2007
Tan, A. M., Samad, O. A., Fischer, T. Z., Zhao, P., Persson, A. K., Waxman, S. G. Maladaptive dendritic spine remodeling contributes to diabetic neuropathic pain. J Neurosci 32: 6795-6807, 2012b
Tan, A. M., Samad, O. A., Liu, S., Bandaru, S., Zhao, P., Waxman, S. G. Burn injury-induced mechanical allodynia is maintained by Rac1-regulated dendritic spine dysgenesis. Exp Neurol 248: 509-519, 2013
Tan, A. M., Stamboulian, S., Chang, Y. W., Zhao, P., Hains, A. B., Waxman, S. G., Hains, B. C. Neuropathic pain memory is maintained by Rac1-regulated dendritic spine remodeling after spinal cord injury. J Neurosci 28: 13173-13183, 2008
Tan, A. M., Waxman, S. G. Dendritic spine dysgenesis in neuropathic pain. Neurosci Lett 601: 54-60, 2015
VanderMolen, K. M., McCulloch, W., Pearce, C. J., Oberlies, N. H. Romidepsin (Istodax, NSC 630176, FR901228, FK228, depsipeptide): a natural product recently approved for cutaneous T-cell lymphoma. J Antibiot (Tokyo) 64: 525-531, 2011
Vorhees, C. V., Williams, M. T. Assessing spatial learning and memory in rodents. ILAR J 55: 310-332, 2014
Wang, S. M., Ooi, L. L., Hui, K. M. Upregulation of Rac GTPase-activating protein 1 is significantly associated with the early recurrence of human hepatocellular carcinoma. Clin Cancer Res 17: 6040-6051, 2011
Wilson, B. M., Cox, C. L. Absence of metabotropic glutamate receptor-mediated plasticity in the neocortex of fragile X mice. Proc Natl Acad Sci USA 104: 2454-2459, 2007
Zhao, P., Hill, M., Liu, S., Chen, L., Bangalore, L., Waxman, S. G., Tan, A. M. Dendritic spine remodeling following early and late Rac1 inhibition after spinal cord injury: evidence for a pain biomarker. J Neurophysiol 115: 2893-2910, 2016

It will be apparent to those skilled in the art that various modifications and variations can be made in the present invention without departing from the scope or spirit of the invention. Other aspects of the invention will be apparent to those skilled in the art from consideration of the specification and practice of the invention disclosed herein. It is intended that the specification and examples be considered as exemplary only, with a true scope and spirit of the invention being indicated by the following claims.

1. A method for treating spasticity in a subject in need thereof, the method comprising administering to the subject an effective amount of a compound having a structure: 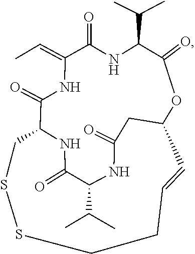 [SMILES: C/C=C1\NC(=O)[C@H]2CSSCC/C=C/[C@H](CC(=O)N[C@H](C(C)C)C(=O)N2)OC(=O)[C@H](C(C)C)NC1=O]
or a pharmaceutically acceptable salt thereof, thereby treating the subject for spasticity.

2. The method of claim 1, wherein spasticity is induced by spinal cord injury (SCI) or multiple sclerosis (MS).

3. The method of claim 1, wherein the subject is not currently undergoing chemotherapy.

4. The method of claim 1, wherein the subject has not previously undergone chemotherapy within the last 7 days.

5. The method of claim 1, wherein the subject has not previously undergone chemotherapy within the last 14 days.

6. The method of claim 1, wherein the subject has not previously undergone chemotherapy within the last month.

7-12. (canceled)

13. The method of claim 1, wherein the effective amount is an amount of from about 0.1 mg/kg to about 0.4 mg/kg.

14. The method of claim 1, wherein the effective amount is an amount of from about 0.2 mg/kg to about 0.3 mg/kg.

15. (canceled)

16. The method of claim 1, wherein the effective amount is administered once per day.

17. The method of claim 1, wherein the effective amount is administered once per day for a period of at least 7 days.

18. The method of claim 1, wherein the effective amount is administered once per day for a period of at least about 14 days.

19. The method of claim 1, wherein administering is via systemic administration.

20-28. (canceled)

29. A method for treating neuropathic pain in a subject in need thereof, the method comprising administering to the subject an effective amount of a compound having a structure: 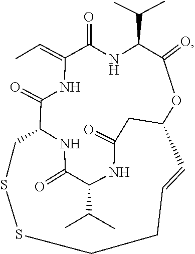 [SMILES: C/C=C1\NC(=O)[C@H]2CSSCC/C=C/[C@H](CC(=O)N[C@H](C(C)C)C(=O)N2)OC(=O)[C@H](C(C)C)NC1=O]
or a pharmaceutically acceptable salt thereof, thereby treating the subject for neuropathic pain.

30. The method of claim 0, wherein the subject has a peripheral nerve injury.

31. The method of claim 0, wherein the peripheral nerve injury is due to amputation or surgical complication or trauma.

32. (canceled)

33. The method of claim 0, wherein the peripheral nerve injury is due to a metabolic disease.

34-40. (canceled)

41. The method of claim 33, wherein the metabolic disease is selected from diabetes and multiple sclerosis (MS).

42. The method of claim 29, wherein the subject is not currently undergoing chemotherapy.

43-44. (canceled)

45. The method of claim 29, wherein the subject has not previously undergone chemotherapy within the last month.

46-67. (canceled)

68. A pharmaceutical composition comprising an effective amount of a compound having a structure: 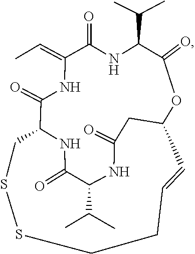 [SMILES: C/C=C1\NC(=O)[C@H]2CSSCC/C=C/[C@H](CC(=O)N[C@H](C(C)C)C(=O)N2)OC(=O)[C@H](C(C)C)NC1=O]
or a pharmaceutically acceptable salt thereof, and an effective amount of one or more of:
(a) an agent known to treat spasticity;
(b) an agent known to treat pain; and
(c) a chemotherapeutic agent, and
a pharmaceutically acceptable carrier.

69-89. (canceled)
